(19) United States
(12) Patent Application Publication
(10) Pub. No.: US 2014/0086827 A1
(43) Pub. Date: Mar. 27, 2014

(54) Serum S100B And Uses Thereof

(71) Applicants: Damir Janigro, Cleveland Heights, OH (US); Nicola Marchi, Cleveland, OH (US)

(72) Inventors: Damir Janigro, Cleveland Heights, OH (US); Nicola Marchi, Cleveland, OH (US)

(73) Assignee: THE CLEVELAND CLINIC FOUNDATION, Cleveland, OH (US)

(21) Appl. No.: 14/116,509

(22) PCT Filed: May 9, 2012
(86) PCT No.: PCT/US2012/037170

§ 371 Date: Nov. 8, 2013

Related U.S. Application Data
(60) Provisional application No. 61/483,974, filed on May 9, 2011.
(60) Provisional application No. 61/522,557, filed on Aug. 11, 2011.

(51) Int. Cl. G01N 33/68; A61K 39/00; A61K 38/18; A61K 51/00; A61K 38/00; A61K 38/43

(52) U.S. Cl. CPC G01N 33/68; A61K 38/00; A61K 38/43; A61K 38/18; A61K 51/00; A61K 39/00
USPC 424/1.11; 514/1.1; 424/94.1; 514/7.6; 424/130.1; 436/501; 435/7.92

(57) ABSTRACT

The invention is directed to a method of assessing blood brain barrier permeability in an individual comprising selectively or specifically detecting a level of S 100BB homodimer in a sample of the individual, and comparing the level of S 100BB homodimer in the sample to a level of S 100B a control. The invention is also directed to methods of determining the effectiveness of a treatment for a neurological disorder wherein blood-brain barrier permeability is present in an individual in need thereof comprising detecting a level of S100B in a sample of the individual undergoing the treatment, and comparing the level of S 100B in the sample to the level of S100B in a sample from the individual obtained prior to treatment. The invention is also directed to a method of detecting a history of blood brain barrier disruption in an individual in need thereof comprising detecting auto [symbol] antibodies directed against S100B in a sample of the individual, wherein the presence of auto-antibodies directed against S100B in the sample indicates that the individual has a history of blood brain barrier disruption.

### RELATED APPLICATIONS

This application claims the benefit of U.S. Provisional Application No. 61/483,974, filed on May 9, 2011 and U.S. Provisional Application No. 61/522,557 file on Aug. 11, 2011. The entire teachings of the above applications are incorporated herein by reference.

### GOVERNMENT SUPPORT

This invention was made with government support under RO1 NS43284, RO1 NS38195, and R21 HD057256 awarded by the National Institutes of Health. The government has certain rights in the invention.

### BACKGROUND OF THE INVENTION

Disruption, particularly uncontrolled disruption, of the blood brain barrier (BBB) is generally believed to be harmful in most circumstances as it can cause influx of potentially dangerous pathogens and inflammatory molecules. However, normal BBB can restrict the entry of potential therapeutic agents into the brain. Therefore, an increased understanding of the molecular mechanisms of the BBB which include the identification of markers of BBB disruption, will provide new avenues of therapeutic intervention for brain disorders.

### SUMMARY OF THE INVENTION

Described herein are studies of S100B, a marker of blood brain-barrier disruption. Experiments described herein further elucidate properties of this marker. There are several proteins in CFS that, when present in serum, are indicators of disruption of the BBB. S100B has been the most useful so far because its levels are normally very low or undetectable in blood (e.g., see U.S. Pat. No. 6,884,591 and U.S. Pat. No. 7,144,708 which are incorporated herein by reference in their entirety). However, several confounding factors interpreting this test have been pointed out and these are addressed herein. The invention described herein is based, in part, on the fact that S100B is present in brain or CFS in different configurations, namely as a monomer, or a dimer, the latter being either heterodimer or homodimer (S100AB or S100BB). Shown herein is that an indicator of blood brain-barrier permeability is the homodimer S100B-S100B.

Current means of detection of S100B at the clinical level do not distinguish between monomeric or dimeric form. This said, there was a commercially available ELISA kit which enabled the detection of any of the above species (Canag).

As shown herein, S100B-S100B dimmer is superior to S100B detection in serum as an indicator of blood brain-barrier permeability. In particular, the dimer provides increased sensitivity and specificity for testing blood brain-barrier permeability (see Pham, N., et al., PLoS ONE, 5(9):e12691 (2010) which is incorporated herein by reference). With the exception of the defunct Canag Ab, commercially available and ELISA or automated kits, all based on the detection of a combination of dimer and monomer forms, can be used.

Accordingly, in one aspect, the invention is directed to a method of assessing blood brain barrier permeability in an individual comprising selectively or specifically detecting a level of S100BB homodimer in a sample of the individual, and comparing the level of S100BB homodimer in the sample to a level of S100B a control. An elevated level of S100BB homodimer in the sample compared to the level of S100BB homodimer in the control, is indicative of blood brain barrier permeability in the individual.

In another aspect, the invention is directed to a method for delivering an agent for delivery to the brain of an individual in need thereof comprising introducing a first agent that opens the blood brain barrier into the individual. The level of S100BB homodimer in a sample of the individual is selectively determined, wherein an elevated level of S100BB homodimer in the sample compared to the level of S100BB homodimer in the control, indicates that the blood brain barrier of the individual is permeable to the agent for delivery to the brain. The agent for delivery to the brain is then introduced to the individual when the blood brain barrier of the individual is permeable, thereby delivering the agent for delivery to the brain of the individual.

In another aspect, the invention is directed to a method of detecting whether a cancer has metastasized to a cancer patient's brain in a patient that has, or is at risk of having, metastasis, comprising detecting a level of S100B in a sample of the cancer patient using a first immunoassay, and detecting a level of S100B in a sample using an immunoassay that differs from the first immunoassay (a second immunoassay; e.g., an immunoassay that is performed at the same time as, or subsequent to, the first immunoassay). The level of S100B in the first and second immunoassays are compared to the level of S100B in a control, wherein if the level of S100B in the first immunoassay and the level of S100B in the second immunoassay are the same as, or lower than the level of S100B in the control then, the metastasis has not spread to the cancer patient's brain.

In yet another aspect, the invention is directed to a method of determining the effectiveness of a treatment for a neurological disorder wherein blood-brain barrier permeability is present in an individual in need thereof comprising detecting a level of S100B in a sample of the individual undergoing the treatment, and comparing the level of S100B in the sample to the level of S100B in a sample from the individual obtained prior to treatment. Decreased levels of S100B in the sample compared to the level of S100B in the sample from the individual obtained prior to treatment indicate that the treatment for a neurological disorder wherein blood-brain barrier permeability is present is effective in the individual.

In another aspect, the invention is directed to a method of determining the effectiveness of a treatment for seizures triggered by blood brain barrier damage in an individual in need thereof comprising detecting a level of S100B in a sample of the individual undergoing the treatment, and comparing the level of S100B in the sample to the level of S100B in a sample from the individual obtained prior to treatment. Decreased levels of S100B in the sample compared to the level of S100B in the sample from the individual obtained prior to treatment indicate that the treatment is effective to treat the seizures in the individual.

In another aspect, the invention is directed to a method of determining the effectiveness of a hypothermia treatment in an individual in need thereof comprising detecting a level of S100B in a sample of the individual undergoing the hypothermia treatment, and comparing the level of S100B in the sample to the level of S100B in a sample from the individual obtained prior to hypothermia treatment. Decreased levels of S100B in the sample compared to the level of S100B in the sample from the individual obtained prior to treatment indicate that the hypothermia treatment is effective to treat the individual.

In another aspect, the invention is directed to a method of detecting a positive outcome for a newborn that has undergone asphyxia during birth comprising detecting a level of S100B in a sample of the newborn at birth, detecting at least one level of S100B in one or more samples of the newborn after birth, and comparing the at least one level of S100B in the sample after birth to the level of S100B in the sample at birth. A decreased level of S100B in the sample after birth compared to the level of S100B at birth indicate a positive outcome for the newborn.

In another aspect, the invention is directed to a method of detecting a sub-concussion in an individual in need thereof comprising detecting a level of S100B in a sample of the individual, and comparing the level of S100B in the sample to a level of S100B a control, wherein elevated levels of S100B in the sample compared to the level of S100B in the control indicate that the individual has a sub-concussion.

In another aspect, the invention is directed to a method of detecting a history of blood brain barrier disruption in an individual in need thereof comprising detecting auto-antibodies directed against S100B in a sample of the individual, wherein the presence of auto-antibodies directed against S100B in the sample indicates that the individual has a history of blood brain barrier disruption.

### BRIEF DESCRIPTION OF THE DRAWINGS

FIGS. 1A-1D: S100B Antibody Comparison. Twelve different types of human tissues were assessed for S100B expression by two different antibodies by Western blot. The OriGene monoclonal antibody was made by immunizing against a synthetic peptide corresponding to residues on the C-terminus of human S100B. The polyclonal Sangtec antibody was raised against the whole human protein, (1A) shows the tissue specific expression level of S100B using the Sangtec-Diasorin antibody and (1B) OriGene antibody after Western blot analysis. Regardless of the antibody used, S100B was found in tissues other than brain. (1C) We quantified and compared the results of the two Western blots obtained by the two different antibodies as well as (1D) this data normalized to brain tissue. The rank order of S100B expression is different depending on the antibody used.

FIGS. 2A-2C: Extracranial Detection of S100B. Extracranial sources of S100B revealed on Western blots do not affect the clinical detection of S100B using the Sangtec-Diasorin immunoassay. (2A) The results of two clinically relevant Sangtec Diasorin immunoassay systems; the fully automated Liaison (x-axis) was compared with the manual LIA-mat assay kit. There was good correlation between the two systems. (2B) A good correlation existed between two automated (Elecsys by Roche Diagnostics and Liaison by DiaSorin) immunoassays for S100B. (2C) The same human tissue protein extracts used for Western blotting previously were analyzed with the Sangtec-Diasorin immunoassay. In contrast to what we found in Western blots, the brain is the main chief expressor detected by this method.

FIG. 3: Hyperosmotic BBB Disruption: Comparison of Various Forms of S100B. The main species of S100B released from BBB disruption is the B-B dimer as measured using the CanAg/Fujirebio ELISA system. The rise in total S100B after BBB opening was 0.011 ng/ml, which was found to be accounted for by the concomitant rise of the B-B dimer. There was virtually no change in the concentration of the A1-B dimer with BBB opening. The concentration of the A1-B dimer was determined by using the CanAg S100A1B EIA solid-phase, two-step, non-competitive immunoassay based on two mouse monoclonal antibodies specific for two different epitopes specifically expressed in S100A1B. The assay thus determines S100A1B with very low cross-reactivity with S100BB or other forms of S100. Similarly, to measure the B-B dimer we used the CanAg S100BB EIA solid-phase, one-step, non-competitive immunoassay based on two mouse monoclonal antibodies specific for two different epitopes specifically expressed in S100BB. This assay thus determines S100BB with very low cross-reactivity with S100A1B or other forms of S100.

FIG. 4: Correlation Between BMI and S100B. Given the expression of S100B in adipocytes, the relationship between fat content and S100B levels was investigated using 200 subjects. No correlation was found between BMI and S100B levels.

FIGS. 5A-5B show PLA Technology (www.olink.com/products-services/duolink/how-use-duolink/pla-probes) that reveals that S100BB is the astrocytic and brain form of S100B. Expression of BB dimer in astrocytes in human brain. A widely utilized technology was used to determine whether BB homodimers are the true reporters of BBBD. The Duolink kits are based on in situ proximity assay (PLA), a technology that extends the capabilities of immunoassays to include direct detection of protein to protein interactions. This enables the study of homodimeric (or heterodimeric) complexes by allowing the use of same (or different) species monoclonal antibodies. The PLA method combines the dual recognition of a probe-targeted assay with a split-reporter approach. A pair of proximity probes (consisting of antibodies to which an oligonucleotide has been conjugated) is used to target the protein of interest. Antibodies are coupled to an oligonucleotide. Upon binding of two PLA to adjacent epitopes on one protein or two molecules in a complex, a connector oligonucleotide hybridizes both proximity probes. It then templates the enzymatic ligation of the oligonucleotides carried by the proximity probes into a full-length molecule. This newly created DNA molecule is a surrogate marker for the detected protein and serves as a template for PCR, utilizing the exponential amplification of PCR to obtain powerful signal amplification that is then visualized by traditional fluorescence methods. FIG. 5A shows PLA Technology (www.olink.com/products-services/duolink/how-use-duolink/pla-probes) that reveals that S100BB is the astrocytic and brain form of S100B. FIG. 5B shows detection of individual B-B homodimers in human brain astrocytes in situ. Note S100BB signal in cell bodies and pericapillary space (astro end feet). ECNu (light DAPI staining) and PeNu (robust DAPI) refer to nuclei of an endothelial cell and a pericyte. Brain tissue was processed for immunocytochemistry and the incubation with anti-S100B antibodies performed as per vendor's suggestion. The same antibody was conjugated with the + and − probe. Thus, any fluorescent signal derived from the reaction between + and − strands was an indication of close proximity of the two antibody-PLA probe complexes. Since both PLA probes were conjugated with the same antibodies and since these antibodies were specific for S100B, the resulting signal could only derive from a juxtaposition of BB homodimers.

FIG. 6 shows that the performance of commercial tests measuring S100B is serum is comparable. However, the negative predictive (NPV) value is maximal when two tests are used in combination. The data refer to samples taken from patients with risk of brain metastases or with ongoing metastatic disease. The negative predictive value refers to contrast enhanced MRI scans used to visualize brain masses. The criteria for detection of metastasis is summarized in the Methods section. The presence of brain metastases was evaluated after contrast injection. Typically, brain metastases presented as highly enhancing lesions with variable degree of perilesional edema. The topographic relationship between metastatic brain tumors and white matter hyperintensities was also studied to emphasize that neoplastic lesions rarely occurred in proximity of primary brain edema presumably pre-existent to the metastatic invasion.

FIG. 7 is a schematic of the two forms of S100B, the monomeric form and homodimeric BB form. The brain protein S100BB is a reporter of blood-brain barrier disruption. In blood, brain S100B is present in dimeric forms; S100BB (homodimer) and S100AB (heterodimer). In addition, other protein-protein interactions involving S100B may occur. As shown herein, the homodimer S100BB is a specific reporter of cerebrovascular damage, representing the brain contribution to serum S100 content. The homodimeric form of S100B is not amenable to further protein binding. Thus, while the S100B monomer may be a marker of blood-brain barrier disruption, the homodimer is preferred since it is a major contributor to the signal measured as total S100B (see FIGS. 5A-5B)

FIGS. 8A-8F: Effects of dexamethasone on pilocarpine-induced SE. (8A) The number of rats experiencing SE was reduced by anti-inflammatory treatments. Data are compared to IL-RA treatments. (8B) When SE developed, its onset was delayed (control vs. IL1ra p=0.03; control vs. Dexa p=0.04). (8C) Twelve hours mortality associated with pilocarpine seizures was decreased by IL-RA and abolished by dexamethasone (p=0.02). To attest the efficacy of treatment on survival, seizures were not stopped using barbiturates. (8D-8F) Examples of EEG recordings show that SE in treated animals was of lesser intensity compared to pilocarpine alone (see also FIGS. 15A-15B). Data are relative to n=15 rats/group. Asterisks: p<0.05, by paired t-test and Fisher test.

FIGS. 9A-9C legend: Time-joint frequency analysis of EEG recordings. (9A-9C) Single asterisk refers to the first seizure episode. The double asterisk shows the maximal electrographic and behavioral seizures observed under any given condition. The actual EEG recordings are also shown. Time joint frequency plots show a reduction of seizure intensity (frequency and amplitude domains, color coded) in treated animals compared to pilocarpine alone. Data shown refer to 2 hours of EEG recordings. See also FIGS. 15A-15B for peak area distribution and instantaneous frequency analysis.

FIGS. 10A-10B: Dexamethasone reduces BBB damage in pilocarpine-treated rats. BBB integrity was assessed by Evans blue (10A) and serum S100β (10B) measurements. Evans blue is an indicator of paracellular leakage while S100β is a surrogate serum marker of the integrity of the cerebrovascular endothelial interface. Both methods revealed a reduction of pilocarpine-induced BBB damage in dexamethasone-pretreated animals (DEXA-PILO). Similar efficacy was previously reported for IL1-RA. The asterisks refers to p<0.05 by paired t-test, n=5 rats per group.

FIGS. 11A-11E: Hematologic and serologic changes after dexamethasone treatment in rats. (11A) Note the drastic decrease of circulating CD3+ T-lymphocytes after dexamethasone treatment. (11B-11C) Note the change in the CD4 and CD8 sub-fractions after treatments. Differences were statistically significant for all the pairs but the ones indicated by n.s. (not significant). (11D) Correlates of T-lymphocyte decreased activation in dexamethasone pretreated animals are summarized as CD4:CD8. (11E) Serum IL1-β levels were reduced by dexamethasone treatment. The asterisks refer to p<0.05 by paired t-test, n=5 animals per group.

FIGS. 12A-12B: Efficacy of dexamethasone and ACTH in drug resistant pediatric epilepsy. Data relative to the efficacy of methyl-prednisolone and hydrocortisone are presented in FIGS. 16A-16D. (12A) A total of 53 treatments were evaluated. Treatments were administered as described in the Methods and Table 4. Seizures were assessed by behavioral and EEG observations. The values reported refer to decrease in seizure burden compared to baseline EEG seizure quantification. T-test was used to assess significance. (12B) Mosaic plot showing the correlation between etiology of epilepsy and likelihood of a response ≧50%. Note that etiology did not always predict response. Noteworthy, dysplasia and other non-encephalopathic diseases responded to the treatments. Cryptogenic seizures were least affected. Bar width is proportional to the number of observations. Colors refer to the response as indicated in the inset.

FIGS. 13A-13B: Radiologic indices of successful treatment with dexamethasone. The seizure reducing effect of dexamethasone (13B) was paralleled by a decrease in FLAIR hyperintensity.

FIGS. 14A-14B: Experimental procedures. (14A) After drug treatment, rats were sacrificed either at SE onset (e.g., to evaluate BBB integrity and FACS-IL-1β analysis) or after 12 hours (e.g., to evaluate EEG changes and mortality). (14B) The total number of rats used and the detailed treatment schedule is provided. IL-RA data see also Methods Section.

FIGS. 15A-15B: Number of events, peak area and instantaneous frequency distribution. EEG traces from pilocarpine alone, dexamethasone, or IL-RA pretreated were analyzed using the event detection routine in ClampFit 9.2. Event threshold was set as 2× baseline across all traces. Analysis of typical traces is provided. Different treatments are indicated by different colors. Pilocarpine SE was characterized by the highest frequencies of events. Spike area (time×amplitude) was also greater compared to dexamethasone (red). (15B) IL-RA pre-treatment lead to qualitatively and quantitatively similar results.

FIG. 16A-16D: Summary of the efficacy of glucocorticosteroids (dexamethasone, methylprednisolone and hydrocortisone) and ACTH in drug resistant pediatric epilepsy. (16A) A total of 92 treatments were evaluated. Treatments were administered as described in the Methods and Table S1. Seizures were assessed by behavioral and EEG observations. The values reported refer to decrease in seizure burden compared to baseline. (16B) Mosaic plot showing the correlation between etiology of epilepsy and likelihood of a response ≧50%, (16C) Although GCs and ACTH were effective across all epileptic syndromes, seizure reduction was more prominent in focal epilepsy patients. (16D) Therapeutic response (set as ≧50%) did not correlate with seizure history.

FIG. 17: Summary of a multivariate analysis of patients' data, serological measurements and drug efficacy. Significant p value (<0.05) is indicated by a red square. Among the variables analyzed the following are here described: 1) age was not a factor influencing GCs or ACTH efficacy; 2) a trend toward significance was observed for the following pairs: efficacy and number of neutrophils, efficacy and number of WBC. A larger population study is required to assess full significance of leukocytes variation in relation to seizure burden and reduction.

FIG. 18: Serum markers were used to monitor the efficacy of clinically relevant maneuvers affecting the BBB. S100B* refers to S100B measured by Diasorin's ELISA. The other species were analyzed with Canag's ELISAs. After a brain hemorrhagic event, serum S100B (monomeric, heterodimeric, homodimeric) levels were increased above the respective thresholds (indicated by dotted lines). S100B (monomeric, heterodimeric, homodimeric) levels were below thresholds in patients who benefited from hypothermia.

FIG. 19: Serum markers were used in the context of paroxysmal pathological events to monitor cerebrovascular status. For example, blood-brain barrier dysfunction occurs in epileptic subjects and contributes to seizure generation. At time of seizures (ictal) S100B is elevated in epileptic patients, indicating cerebrovascular damage. The Diasorin ELISA was used to measure S100B.

FIG. 20A: The reduction of sign and symptoms after administration of a given brain drug may elicit days or weeks after the beginning of the therapy. Serum markers (Diasorin ELISA was used to measure S100B) were capable of indicating the reduction of the bio-pathological substrate (i.e. restoration of cerebrovascular integrity) that occurs prior to amelioration of signs and symptoms in epileptics rats and subjects. For example, S100B blood levels decreased in an animal model of epilepsy where restoration of the cerebrovascular function (as obtained by anti-inflammatory drugs) led to seizure reduction and to a long-term improved outcome (e.g., decrease mortality). Improved blood-brain barrier function and decrease of seizure frequency was observed in epileptic patients receiving anti-inflammatory therapy (dexamethasone, or dexa; prednisolone and hydrocortisone were also used and had comparable effects). Currently changes in cerebrovascular function can be monitored only using T2-FLAIR or contrast agents such as gadolinium. Note the disappearance of sub-cortical hyperintensity in the post-treatment stage. The latter was associated with a significant long-term disappearance of sign and symptoms and improved outcome.

FIG. 20B: Improved blood-brain barrier function and decrease of seizure frequency was observed in epileptic patients receiving anti-inflammatory therapy (dexamethasone, or dexa; prednisolone and hydrocortisone were also used and had comparable effects). Currently changes in cerebrovascular function can be monitored only using T2-FLAIR or contrast agents such as gadolinium. Note the disappearance of sub-cortical hyperintensity in the post-treatment stage. The latter was associated with a significant long-term disappearance of sign and symptoms and improved outcome.

FIG. 21: Protein markers (the Diasorin ELISA was used to measure total S100B) were used to predict clinical, long-term outcome after pathological events affecting the cerebrovasculature and the brain. The data show that urine markers can be used to predict long-term outcome resulting from an acute cerebrovascular event; this is significant in a population where venous blood draw is difficult and often unacceptable due to parents' concerns. S100B levels were elevated in newborn with poor outcome following perinatal asphyxia. S100B levels in newborn where signs resolved or that had a normal birth are also shown. Perinatal asphyxia was associated with alteration of the cerebral blood flow and cerebrovascular damage.

FIG. 22: Serum markers (Diasorin ELISA was used to measure S100B) can be used to assess long-term outcome after traumatic brain injury. Serum markers can be used to segregate subjects at risk of developing brain impairment. Serum markers of BBB damage indicate subjects at risk for brain damage after a severe hit. S100B blood levels were elevated in football players experiencing significant head hits during a game (as detected by film review and post-game interview). Note that S100B serum levels did not increase in players who did not experience head hits. The values in Y axis indicate S100 Bpost game-100 Bpre-game which refers to samples taken 24 hours before the game and within 1 hour post-game. Head trauma and concussion are associated with cerebrovascular dysfunction leading to the development of cognitive decline and brain injury.

FIG. 23: Temporal relationship between S100B levels in brain and serum, and the production of auto-antibodies. The brain is a partially “immune privileged” site, meaning that the body may not know about all of the brain antigens. Following BBB disruption, the body may wrongly mount an attack on the “foreign” antigen which in this case astrocytes expressing S100B. The fact that S100B expressing astrocytes are primarily concentrated in gray matter may influence the downstream clinical consequences of this autoimmune response.

FIG. 24: Anti-S100B autoimmunity after repeated BBBD. A) Following ablation of tumor, patients were followed for several months. Note transient elevations in S100B and the significant (p=0.02) increase in anti-S100B Ab levels (boxed symbols red y axis). Each line refers to a patient; note that the red scale refers to autoimmune signals and that the same scale is shared in B. B) Serum samples from patients undergoing repeated, controlled iatrogenic BBB disruptions. Note the increase in autoimmune signal over time.

FIG. 25A shows a graph of autoimmune titer in control vs. football players after the 2011 season. 8 players and as many healthy volunteers were enrolled. Note the significant difference in baseline, unstimulated levels of autoantibodies in football players, and a graph of time-dependent decline of auto-S100B antibodies in serum of 4 players tested at different intervals after minor traumatic brain injury (last concussion diagnosed by team doctors).

FIG. 25B is a graph showing correlation between serum S100B increase and auto-immunity in football players. Note that repetitive elevation in serum S100B levels (indicated in the Y axis by a summation value, ng/ml) triggers an auto-immune reaction against S100B (X axis). Presence of Auto-Ab against S100B (or other brain proteins) in serum is a pathological hall mark of brain disorders. These data demonstrated that S100B elevation predict long term pathological reactions (e.g. auto-immunity against brain protein). These data also show that a combinatory measurement of S100B and its Auto-Ab can be used to predict subjects at risk for cognitive decline.

### DETAILED DESCRIPTION OF THE INVENTION

S100B, established as prevalent protein of the central nervous system, is a peripheral biomarker for blood-brain barrier disruption and often also a marker of brain injury. However, reports of extracranial sources of S100B, especially from adipose tissue, may confound its interpretation in the clinical setting. Described herein is the characterization of the tissue specificity of S100B and the assessment of how extracranial sources of S100B affect serum levels. Specifically, as described herein, the extracranial sources of S100B were determined by analyzing nine different types of human tissues by ELISA and Western blot. In addition, brain and adipose tissue were further analyzed by mass spectrometry. A study of 200 subjects was undertaken to determine the relationship between body mass index (BMI) and S100B serum levels. The levels of S100B homo- and heterodimers in serum were measured quantitatively after blood-brain barrier disruption. Analysis of human tissues by ELISA and Western blot revealed variable levels of S100B expression. By ELISA, brain tissue expressed the highest S100B levels. Similarly, Western blot measurements revealed that brain tissue expressed high levels of S100B but comparable levels were found in skeletal muscle. Mass spectrometry of brain and adipose tissue confirmed the presence of S100B but also revealed the presence of S100A1. The analysis of 200 subjects revealed no statistically significant relationship between BMI and S100B levels. Moreover, shown herein is that the main species of S100B released from the brain was the B-B homodimer. The results show that extracranial sources of S100B do not affect serum levels. Thus, the diagnostic value of S100B and its negative predictive value in neurological diseases in intact subjects (without traumatic brain or bodily injury from accident or surgery) are not compromised in the clinical setting.

Also described herein is the investigation of the targeting pro-inflammatory events to reduce seizures and the ability to evaluate the efficacy of such treatments using S100B. Experimentally, antagonism of inflammatory processes and of blood-brain barrier (BBB) damage has been demonstrated to be beneficial in reducing status epilepticus (SE). Clinically, a role of inflammation in the pathophysiology of drug resistant epilepsies is suspected. However, the use anti-inflammatory drug such as glucocorticosteroids (GCs) is limited to selected pediatric epileptic syndromes and spasms. Lack of animal data may be one of the reasons for the limited use of GCs in epilepsy. The effect of the CG dexamethasone in reducing the onset and the severity of pilocarpine SE in rats was evaluated. BBB integrity was assessed by measuring serum S100β and Evans Blue brain extravasation. Electrophysiological monitoring and hematologic measurements (WBCs and IL-1β) were performed. The effect of add on dexamethasone treatment on a population of pediatric patients affected by drug resistant epilepsy was reviewed. Subjects affected by West, Landau-Kleffner or Lennox-Gastaut syndromes and Rasmussen encephalitis, known to respond to GCs or adrenocorticotropic hormone (ACTH), were excluded. The effect of two additional GCs, methylprednisolone and hydrocortisone, was also reviewed in this population. When dexamethasone treatment preceded exposure to the convulsive agent pilocarpine, the number of rats developing status epilepticus (SE) was reduced. When SE developed, the time-to-onset was significantly delayed compared to pilocarpine alone and mortality associated with pilocarpine-SE was abolished. Dexamethasone significantly protected the BBB from damage. The clinical study included pediatric drug resistant epileptic subjects receiving add on GC treatments. Decreased seizure frequency (≧50%) or interruption of status epilepticus was observed in the majority of the subjects, regardless of the underlying pathology. The experimental results point to a seizure-reducing effect of dexamethasone. The mechanism encompasses improvement of BBB integrity. The results also indicate that add on GCs could be of efficacy in controlling pediatric drug resistant seizures (Marchi, N., et al., PLoS ONE, 6(3):e18200 (2011) which is incorporated herein by reference).

In addition to seizures, S100B can be used as a prognostic indicator for a variety of disorders associated with blood brain barrier dysfunction and the treatment thereof.

Finally, also shown herein is that autoantibodies directed against S100B, S100BB, S100AB or a combination thereof can be detected in samples form individuals with a history of blood brain barrier disruption.

Accordingly, in one aspect, the invention is directed to a method of assessing blood brain barrier permeability in an individual comprising selectively or specifically detecting a level of S100BB homodimer in a sample of the individual, and comparing the level of S100BB homodimer in the sample to a level of S100B a control. An elevated level of S100BB homodimer in the sample compared to the level of S100BB homodimer in the control, is indicative of blood brain barrier permeability in the individual.

The blood brain barrier is a naturally occurring barrier created by the modification of brain capillaries (as by reduction in fenestration and formation of tight cell-to-cell contacts) that prevents many substances from leaving the blood and crossing the capillary walls into the brain tissues. “S100B” is a prevalent protein of the central nervous system which is used as a peripheral biomarker for blood-brain barrier disruption and as a marker of brain injury. S100B can occur in a monomeric form, referred to herein as S100B or S100B monomer; a homodimeric form referred to herein as S100BB homodimer, S100BB, B-B homodimer, or S100B-B; or a heterodimeric form referred to herein as S100AB heterodimer, S100AB, A-B heterodimer, or S100A-B. Thus, depending on the context, the term “S100B” can be used to refer to the S100B monomer, the S100B homodimer, the S100B heterodimer, or the combined total of S100B monomer, S100BB homodimer and S100AB heterodimer (total S100B).

In the methods described herein a (one or more) level of S100B is measured. Unless otherwise specified, the level of S100B measured can be the level of S100B monomer, S100BB homodimer, S100AB, total S100B or a combination thereof.

In some embodiments of the methods described herein, one or more of the S100B proteins is selectively measured. Selective detection of one or more S100B proteins refers to the ability to detect one or more S100B proteins in a sample to the exclusion of other forms of one or more S100B proteins and other molecules in a sample. Thus, for example, selective detection of S100BB homodimer refers to the ability to detect the S100BB heterodimer in a sample to the exclusion of other forms of the S100B protein (e.g., to the exclusion of S100B monomer, S100AB heterodimer) and other molecules (e.g., protein) in a sample.

As will be appreciated by those of skill in the art, the methods described herein can further comprise detecting one or more levels of one or more markers of neuronal distress. Examples of markers of neuronal distress include Ubiquitin C-terminal hydrolase 1, NSE, GFAP, tau protein, beta trace protein, cystatin C.

The method of assessing blood brain barrier permeability in an individual can further comprising detecting whether auto-antibodies directed against S100B, S100BB, S100AB or a combination thereof are present in a sample (e.g., the same sample used to detect S100BB homodimer, or a different sample) of the individual.

The detection of the S100B protein (e.g., S100BB homodimer) and/or autoantibodies directed against S100B, S100BB, S100AB or a combination thereof can be detected at the same time or at different times and at one or more time periods as determined necessary by one of skill in the art. Thus, in the methods described herein, the S100BB homodimer and/or autoantibodies directed against S100B, S100BB, S100AB or a combination thereof can be detected in a single sample or in multiple samples (samplings) and/or over a period of time, as needed.

In another aspect, the invention is directed to a method for delivering an agent for delivery to the brain of an individual in need thereof comprising introducing an agent (a first agent) or condition that opens (transiently opens) the blood brain barrier into the individual. The level of S100BB homodimer in a sample of the individual is selectively determined, wherein an elevated level of S100BB homodimer in the sample compared to the level of S100BB homodimer in the control, indicates that the blood brain barrier of the individual is permeable to the agent for delivery to the brain. The agent for delivery to the brain (a second agent) is then introduced to the individual when the blood brain barrier of the individual is permeable, thereby delivering the agent for delivery to the brain of the individual.

Agents which cause the blood brain barrier to open are known to those of skill in the art. Examples of such agents include hyperosmolar osmotic agents such as mannitol (intraarterial injection), bradikinin and its analog RPM-7 (B2 receptor agonist), and alkilglycerola (Stamataovic, S., et al., Current Neuropharmacology, 6:179-192 (2008)). In some instances the blood brain barrier of the individual will be open due to conditions to which the individual is exposed or is undergoing, such as inflammation or exposure of BBB to radiotherapy (20-30 Gy) (Stamataovic, S., et al., Current Neuropharmacology, 6:179-192 (2008)).

As will be appreciated by those of skill in the art, a variety of agents can be delivered to the brain using the methods described herein. Examples of agents include a contrast agent, a neuropharmacologic agent, a neuroactive peptides, a protein, an enzyme, a gene therapy agent, a neuroprotective factor, a growth factor, a biogenic amine, a trophic factor to any of brain and spinal transplants, an immunoreactive proteins, a receptor binding protein, a radioactive agent, an antibody, a cytotoxin or a combination thereof.

Methods for delivering the agent that is to be delivered to the brain are also apparent to those of skill in the art. In a particular aspect, the agent for delivery to the brain is introduced into the individual's bloodstream in a vicinity of the individual's brain. For example, the agent can be delivered via intra-carotid and/or intranasal injection.

In another aspect, the invention is directed to a method of detecting whether a cancer has metastasized to a cancer patient's brain in a patient that has, or is at risk of having, metastasis, comprising detecting a level of S100B in a sample of the cancer patient using a first immunoassay, and detecting a level of S100B in a sample using an immunoassay that differs from the first immunoassay (a second immunoassay; e.g., an immunoassay that is performed at the same time as, or subsequent to, the first immunoassay). The level of S100B in the first and second immunoassays are compared to the level of S100B in a control, wherein if the level of S100B in the first immunoassay and the level of S100B in the second immunoassay are the same as, or lower than the level of S100B in the control then, the metastasis has not spread to the cancer patient's brain. In one aspect, the level of S100B monomer, S100BB homodimer, S100AB or total S100B or a combination thereof is detected. In another aspect, the level of S100BB homodimer is selectively detected.

In yet another aspect, the invention is directed to a method of determining the effectiveness of a treatment for a neurological disorder wherein blood-brain barrier permeability is present in an individual in need thereof comprising detecting a level of S100B in a sample of the individual undergoing the treatment, and comparing the level of S100B in the sample to the level of S100B in a sample from the individual obtained prior to treatment. Decreased levels of S100B in the sample compared to the level of S100B in the sample from the individual obtained prior to treatment indicate that the treatment for a neurological disorder wherein blood-brain barrier permeability is present is effective in the individual. In one aspect, the level of S100B monomer, S100BB homodimer, S100AB or total S100B or a combination thereof is detected. In another aspect, the level of S100BB homodimer is selectively detected. Examples of a neurological disorder wherein blood-brain barrier permeability is present includes multiple sclerosis, tumors, psychiatric disorders and the like.

In another aspect, the invention is directed to a method of determining the effectiveness of a treatment for seizures triggered by blood brain barrier damage in an individual in need thereof comprising detecting a level of S100B in a sample of the individual undergoing the treatment, and comparing the level of S100B in the sample to the level of S100B in a sample from the individual obtained prior to treatment. Decreased levels of S100B in the sample compared to the level of S100B in the sample from the individual obtained prior to treatment indicate that the treatment is effective to treat the seizures in the individual. In one aspect, the level of S100B monomer, S100BB homodimer, S100AB or total S100B or a combination thereof is detected. In another aspect, the level of S100B13 homodimer is selectively detected.

In another aspect, the invention is directed to a method of determining the effectiveness of a hypothermia treatment in an individual in need thereof. As will be appreciated by those of skill in the art, hypothermia is administered to treat a variety of reasons such as to treat ischemic-hemorrhagic stroke, to mitigate seizures or during a surgical cardiac procedure in the individual. The method comprises detecting a level of S100B′ in a sample of the individual undergoing the hypothermia treatment, and comparing the level of S100B in the sample to the level of S100B in a sample from the individual obtained prior to hypothermia treatment. Decreased levels of S100B in the sample compared to the level of S100B in the sample from the individual obtained prior to treatment indicate that the hypothermia treatment is effective to treat the individual. In one aspect, the level of S100B monomer, S100BB homodimer, S100AB or total S100B or a combination thereof is detected. In another aspect, the level of S100BB homodimer is selectively detected.

In another aspect, the invention is directed to a method of detecting a positive outcome for a newborn that has undergone asphyxia during birth comprising detecting a level of S100B in a sample of the newborn at birth, detecting at least one level of S100B in one or more samples of the newborn after birth, and comparing the at least one level of S100B in the sample after birth to the level of S100B in the sample at birth. A decreased level of S100B in the sample after birth compared to the level of S100B at birth indicate a positive outcome for the newborn. In one aspect, the level of S100B monomer, S100BB homodimer, S100AB or total S100B or a combination thereof is detected. In another aspect, the level of S100BB homodimer is selectively detected.

In another aspect, the invention is directed to a method of detecting a sub-concussion in an individual in need thereof comprising detecting a level of S100B in a sample of the individual, and comparing the level of S100B in the sample to a level of S100B a control, wherein elevated levels of S100B in the sample compared to the level of S100B in the control indicate that the individual has a sub-concussion. In one aspect, the level of S100B monomer, S100BB homodimer, S100AB or total S100B or a combination thereof is detected. In another aspect, the level of S100BB homodimer is selectively detected. In yet another aspect, the individual has had one or more concussions, sub-concussions, seizures or a combination thereof.

In another aspect, the invention is directed to a method of detecting a history of blood brain barrier disruption in an individual in need thereof comprising detecting auto-antibodies directed against S100B in a sample of the individual, wherein the presence of auto-antibodies directed against S100B in the sample indicates that the individual has a history of blood brain barrier disruption. In one aspect, the auto-antibodies are directed against S100B monomer, S100BB heterodimer, S100AB heterodimer or a combination thereof. The method can further comprise detecting a level of S100B in a sample of the individual wherein elevated levels of S100B in the sample compared to the level of S100B in the control further indicates that the individual has a history of blood brain barrier disruption. In one aspect, the level of S100B monomer, S100BB homodimer, S100AB or total S100B or a combination thereof is detected. In another aspect, the level of S100BB homodimer is selectively detected. In particular aspects, the individual has ongoing blood brain barrier disruption, has an increased risk for degenerative brain disease, or has had one or more concussion, sub-concussions, seizures or a combination thereof.

In a particular embodiment, the invention is directed to a method of detecting a history of blood brain barrier disruption in an individual in need thereof comprising detecting S100B and auto-antibodies directed against S100B in a (one or more) sample of the individual, wherein the presence of S100B and auto-antibodies directed against S100B in the sample indicates that the individual has a history of blood brain barrier disruption. In one aspect, the level of S100B monomer, S100BB homodimer, S100AB or total S100B or a combination thereof is detected. In another aspect, the level of S100BB homodimer is selectively detected. In yet another aspect, the auto-antibodies are directed against S100B monomer, S100BB heterodimer, S100AB heterodimer or a combination thereof. In particular aspects, the individual has ongoing blood brain barrier disruption, has an increased risk for degenerative brain disease, or has had one or more concussion, sub-concussions, seizures or a combination thereof.

The methods described herein can further comprise obtaining a sample from the individual (e.g., prior to treatment). The methods can also comprise contacting the sample with an agent that detects S100B (e.g., S100B monomer, S100BB homodimer, S100AB or total S100B or a combination thereof) or auto-antibodies directed against S100B), thereby producing a combination or mixture. The combination can be maintained under conditions which allow detection of the S100B or auto-antibodies directed against S100B.

As described herein, in some aspects of the invention, decreased levels of S100B indicate the effectiveness of a treatment. In a particular embodiment, a decrease of about 10%, 20%, 30%, 40% or 50% in the level of S100B (e.g., S100B monomer, S100BB homodimer, S100AB or total S100B or a combination thereof) detected in the sample is indicative of the effectiveness of a treatment.

Detection of the one or more forms of S100B can be performed using a variety of methods known to those of skill in the art. The S100B (e.g., S100B monomer, S100BB homodimer, S100AB or total S100B or a combination thereof) molecules can be detected alone or in a complex with another molecule. In one aspect, the S100B is detected using mass spectrometry or a proteomic test based on immunodetection. In other aspects, the S100B is detected using an immunoassay such as an immunoprecipitation assay. In particular aspects, a sample obtained from an individual can be contacted with an agent that captures (e.g., binds to) one or more forms of S100B (e, g, such an agent can be added to a sample obtained from an individual), thereby faulting a combination or mixture. The combination or mixture can be maintained under conditions in which the agent captures the one or more forms of S100B in the sample, thereby forming a complex between the one or more forms of the S100B and the capture agent. In a specific embodiment, the capture agent is an antibody or antigen binding fragment thereof that specifically binds to, or has a binding affinity for, one or more forms of S100B (e.g., an antibody that specifically binds to one or more forms of S100B monomer, S100BB homodimer, S100AB heterodimer or a combination thereof).

As used herein, the terms “specific”, “selective”, “specifically”, “selectively” when referring to a capture agent such as an antibody-antigen interaction, is used to indicate that the capture agent (e.g., antibody) can selectively bind to one or more forms of S100B. In particular aspects, the capture agent is an antibody. In one embodiment, the antibody selectively binds to all or a portion of S100B monomer. In another embodiment, the antibody selectively binds to all or a portion of S100BB homodimer. In yet another embodiment, the antibody specifically binds to all or a portion of S100AB heterodimer.

An antibody that is specific for one or more forms of S100B is a molecule that selectively binds to one or more forms of S100B (e.g., selectively binds to S100BB homodimer) but does not substantially bind to other forms of S100B (e.g., S100B monomer, S100AB heterodimer) or other molecules in a sample, e.g., in a biological sample that contains one or more forms of S100B. The term “antibody,” as used herein, refers to an immunoglobulin or a part thereof (e.g., an antigen binding fragment thereof), and encompasses any polypeptide comprising an antigen-binding site regardless of the source, method of production, and other characteristics. The term includes but is not limited to polyclonal, monoclonal, monospecific, polyspecific, humanized, human, single-chain, chimeric, synthetic, recombinant, hybrid, mutated, conjugated and CDR-grafted antibodies. The term “antigen-binding site” refers to the part of an antibody molecule that comprises the area specifically binding to or complementary to, a part or all of an antigen. An antigen-binding site may comprise an antibody light chain variable region (VL) and an antibody heavy chain variable region (VH). An antigen-binding site may be provided by one or more antibody variable domains (e.g., an Fd antibody fragment consisting of a VH domain, an Fv antibody fragment consisting of a VH domain and a VL domain, or an scFv antibody fragment consisting of a VH domain and a VL domain joined by a linker). For example, the term “anti-S100B monomer antibody,” “antibody against S100B monomer,” refers to any antibody that specifically binds to at least one epitope of S100B monomer; the term “anti-S100BB homodimer antibody,” “antibody against S100BB homodimer,” refers to any antibody that specifically binds to at least one epitope of S100BB homodimer; and the term “anti-S100AB heterodimer antibody,” “antibody against S100AB heterodimer,” refers to any antibody that specifically binds to at least one epitope of S100AB heterodimer.

The various antibodies and portions thereof can be produced using known techniques (Kohler and Milstein, Nature 256:495-497 (1975); Current Protocols in Immunology, Coligan et al., (eds.) John Wiley & Sons, Inc., New York, N.Y. (1994); Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1; Newman, R. et al., BioTechnology, 10: 1455-1460 (1992); Ladner et al., U.S. Pat. No. 4,946,778; Bird, R. E. et al., Science, 242: 423-426 (1988)).

As will also be appreciated by those of skill in the art, a variety of methods can be used to detect auto-antibodies directed against S100B. Examples of such assays include an enzyme-linked immunosorbent assay (ELISA) and immunofluoresence (e.g., indirect immunofluorescence).

As described herein, in the methods the amount of S100B and/or autoantibodies directed against S100B in the individual (e.g., a sample of the individual) can be compared to the amount of S100B and/or autoantibodies directed against S100B in a suitable control. As will be appreciated by one of skill in the art, there are a variety of suitable controls that can be used in the methods described herein. For example, the control can be a sample from an individual that does not have a permeable BBB, a BBB disruption, a brain disorder and/or a brain trauma.

As will also be appreciated by those of skill in the art, any suitable biological sample can be used in the methods described herein. Examples of biological samples include urine, blood, serum, spinal fluid (e.g. cerebral spinal fluid), lymph, and tissue. The sample can be obtained from the individual and/or analyzed for the presence of S100B and/or autoantibodies directed against S100B using known methods.

The amount of S1000B and/or autoantibodies directed against S100B in the sample can be compared to the amount of S100B or autoantibodies directed against S100B in a suitable control. Suitable controls include a previous reading of S100B and/or autoantibodies directed against S100B in the individual prior to BBB permeability or disruption (e.g., a reading obtained from the same individual prior to metastasis of a cancer to the brain, an epileptic seizure, a brain trauma, a brain disorder, a sub-concussion, a concussion), readings (from one or more individuals with normal BBB, and of a known standard.

As used herein, the term “individual” refers to an “animal” which includes mammals, as well as other animals, vertebrate and invertebrate (e.g., birds, fish, reptiles, insects (e.g., Drosophila species), mollusks (e.g., Aplysia). In a particular embodiment, the animal is a mammal. The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include primates (e.g., humans, monkeys, chimpanzees), rodents (e.g., rats, mice, guinea pigs), ruminents (e.g., cows, pigs, horses) felines and canines.

### Exemplification

### Antibodies

| Commercial | | Cross reactivity | Lowest Level of |
|---|---|---|---|
| origin | Target | with S100A1 | Sensitivity |
| Diasorin⁺ | S100B | Minimal | ~0.02 ng/ml |
| Canag⁺⁺ | S100A1B + | Recognizes | ~0.02 ng/ml |
| | S100BB | S100A1 | |
| | | No cross- | |
| | | reactivity with | |
| | | other forms of | |
| | | S100 | |
| Canag | specific for two | Not relevant; | ND |
| | different | very low cross- | |
| | epitopes | reactivity with | |
| | specifically | S100BB or other | |
| | expressed in | forms of S100 | |
| | S100A1B | | |
| Canag | specific for two | Not relevant; | ND |
| | different | very low cross- | |
| | epitopes | reactivity with | |
| | specifically | S100A1B or | |
| | expressed in | other forms of | |
| | S100BB. | S100 | |

⁺Originally Sangtec Medical, Sweden
⁺⁺Now Fujirebio Diagnostics

### Example 1

### Extracranial Sources of S100B do not Affect Serum Levels

### Methods

### Human Subjects

All studies were performed in accordance with the Declaration of Helsinki and written approval by the IRB Committee at the Cleveland Clinic. All samples (blood and tissue) were obtained by written informed consent and given a coded identifier to retain subject anonymity. Tissue samples were obtained as surplus tissue from various medical procedures conducted at the Cleveland Clinic, generously provided by the department of Pathology. For the purpose of this study we analyzed 200 subjects, consisting of 155 patients and 45 controls (Table 1). Note that the patient population comprised of individuals with a broad spectrum of ages, races, and pathologies, which ranges from psychiatric disorders to brain tumors. A large number of volunteers without any pathology or psychiatric condition, as detected by a psychiatrist delivering a brief psychiatric exam, were also recruited. Samples were analyzed by different techniques, the details of which are presented in the Methods Section and below. Tissue protein contents was analyzed by antibody-dependent assays or by mass spectrometry (MS). When possible, two antibodies were used for the detection of different antigen regions in the S100B protein. BMI (body mass index) was calculated based on the weight (in kilograms) divided by the square of the patient's height (in meters) taken from the medical record.

### BBB Disruption

The Cleveland Clinic Brain Tumor Institute provides a treatment called blood-brain barrier disruption for primary CNS lymphomas. All procedures were performed after informed consent was obtained using protocols approved by the Cleveland Clinic Foundation IRB. In this protocol, intra-arterial mannitol (1.4 M) is administered via a carotid or vertebral artery, and BBB disruption was confirmed by contrast CT immediately after chemotherapy. The details are described elsewhere [Marchi, N., et al., Epilepsia, 48:732-742 (2007)].

### Tissue Protein Extraction

Proteins were extracted from various tissues using the Millipore Total Protein Extraction Kit (Chemicon subsidiary, Temecula, Calif.). Briefly, tissues were weighed, chopped into small pieces, and kept on dry ice. Then 1×TM buffer [13 mL of HEPES (pH 7.9), MgCl2, KCl, EDTA, sucrose, glycerol, sodium deoxycholate, NP-40, sodium orthovanadate] and a protease inhibitor cocktail was added to each tissues at 2.5 mL per gram of tissue and put on ice for 5 minutes. The tissue was homogenized for 20 seconds and then put on dry ice for 15 seconds. This cycle was repeated 3 times. The homogenized tissues were rotated at 4° C. for 20 minutes and centrifuged at 11,000 rpm at 4° C. for 20 minutes. The supernatant was collected and stored at −80° C. until analyzed further.

### Western Blots

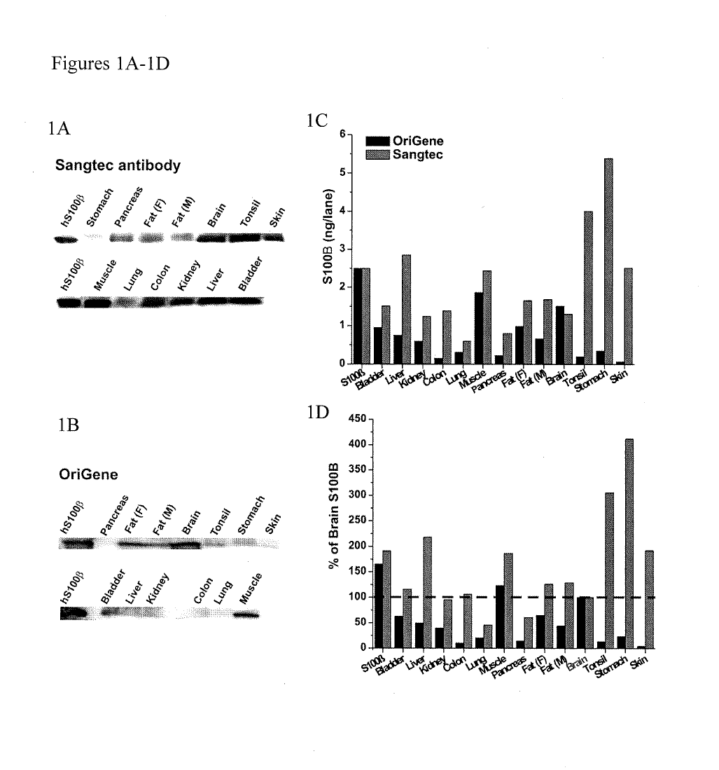

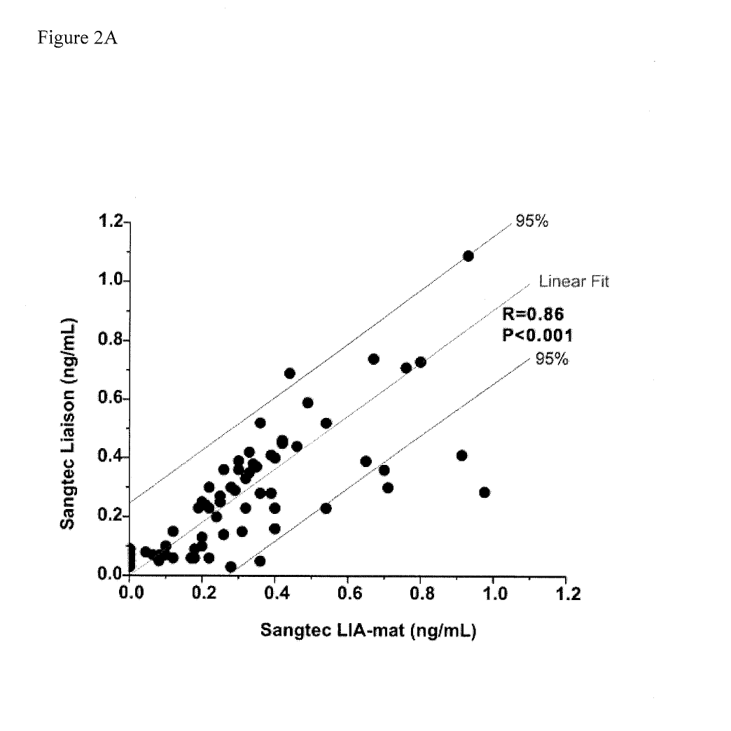

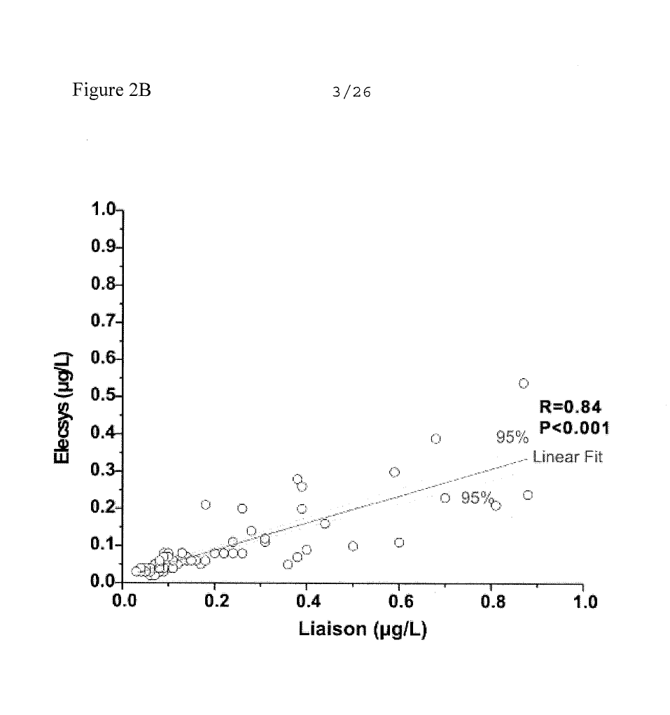

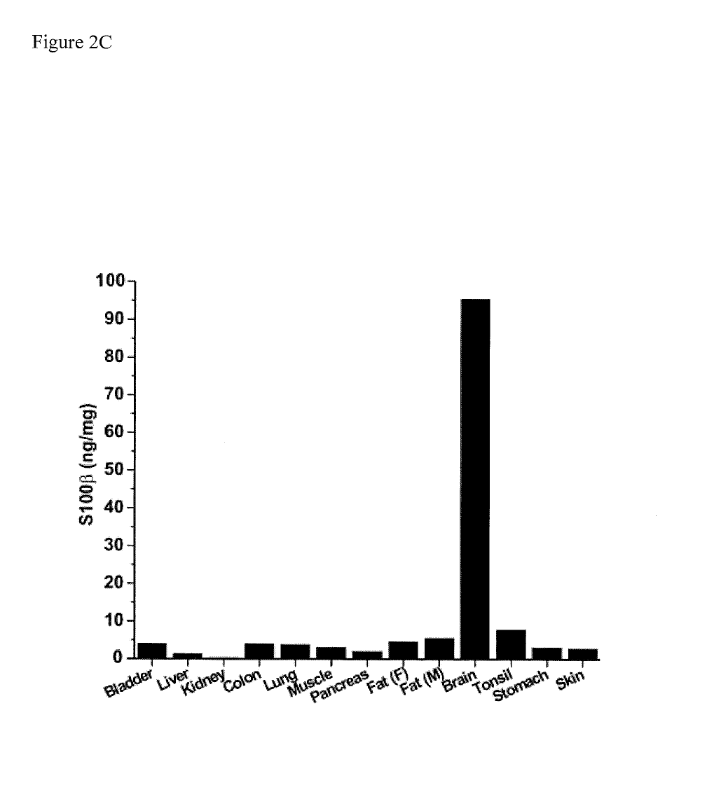

Protein concentration was determined by the Bradford assay method (Bio-Rad, Hercules, Calif.). Total proteins (50 μg/lane) were separated on 10-20% polyacrylamide gels with SDS-PAGE at 80 V and transferred onto a polyvinylidene difluoride membrane (Millipore Corp., Bedford, Mass.) by electroblotting at 100 V of constant voltage for 1 hour. After blocking with TBST and milk (Tris-buffered saline, 0.05% milk powder, and 0.05% Tween 20) for at least 2 hours, the membrane was probed overnight at 4° C. either with the Sangtec-Diasorin or OriGene S100B primary antibody (1:1000). The OriGene monoclonal antibody was made by immunizing against a synthetic peptide corresponding to residues on the C-terminus of human S100B. The polyclonal Sangtec antibody was raised against the whole human protein. These antibodies were selected because they target different regions of the S100B protein (see legend of FIG. 1). The Sangtec antibody was also chosen for Western blot analysis because it constitutes the capture antibody of the LIAISON kit (FIGS. 2A, 2B). After a series of washes, the membrane was incubated with secondary horseradish peroxidase-conjugated anti-goat IgG antibody (rabbit or rat) for 2 hours. Western blots were visualized by enhanced chemiluminescence reagent (ECL Plus, Amersham Biosciences, Piscataway, N.J.). The dilution for the Sangtec antibody, which is not commercially available, was derived from a previous study [Fazio, V., et al., Ann Thorac Surg, 78:46-52 (2004)],

### Relative Density on Western Blots

Western blots were scanned on a scanner interfaced with a PC using HP Precision Scan Pro 3.02 analysis software (Hewlett-Packard Co., Palo Alto, Calif.). The scanned grayscale images were saved in an uncompressed TIFF format and further analyzed using software specifically designed for measuring grayscale image density, developed by Nonlinear USA, Inc. (Durham, N.C.) Phoretix™ ID (Version 2003.01).

### Immunoassay Determination

The colorimetric immunosorbent assay, Sangtec® 100 ELISA, by DiaSorin, Inc. (Stillwater, Minn.) was used to quantify S100B. The limit of detection is 0.03 ng/mL. The Canag/Fujirebio system by Fujirebio Diagnostics, Inc. (Tokyo, Japan) was also used to measure various mono- and hetero-dimers of S100B. The Elecsys system (Roche Diagnostics, Indianapolis, Ind.) was used as well for the purpose of measuring S100B.

### Mass Spectrometry

A LC-MS system Finnigan LCQ-Deca ion trap mass spectrometer system with a Protana microelectrospray ion source interfaced to a self-packed 10 cm×75 um id Phenomenex Jupiter C18 reversed-phase capillary chromatography column was used. Data were analyzed by using all CID spectra collected in the experiment to search the National Center of Biotechnology Information (NCBI) non-redundant database with the search program TurboSequest. All matching spectra were verified by manual interpretation. The interpretation process was also aided by using the programs Mascot and Fasta to perform additional searches, as needed.

### Statistical Methods

Data are presented as mean±standard deviation (SD). JMP® 8.0 (Cary, N.C.) was used for statistical analysis. Correlation plots were produced using statistical software by the Origin Lab Corporation (versions 7.0 and higher, Northampton, Mass.) to calculate correlation coefficients (R) and 95% confidence limits. Significant difference or correlation was assessed by P-values of <0.05, calculated using the Student's t-statistic.

### Results

A study of whether tissue sources, outside the brain, contribute significantly to serum levels of S100B is described herein. In this study, the expression of S100B in human tissue was characterized by Western blot using two different antibodies and this data was corroborated with mass spectrometry. How expression in these tissues affects the current clinical methods for detection of S100 was also assessed using the LIAISON® S100 or LIAMAT systems (Sangtec-Diasorin), and the Elecsys system (Roche Diagnostics). 200 subjects were also analyzed to determine if adiposity or fat S100B affected serum levels.

S100B protein content was first analyzed in a variety of tissue samples (Table 2). Western blot analysis revealed that, regardless of the antibody used, S100B is found in tissues other than brain. The results in FIGS. 1A and 1B show protein signals obtained with the Sangtec or OriGene antibody. The results presented thus far were obtained with two different antibodies directed towards the same antigen, yet these results were not quantitatively consistent. To rule out that the measured signal may be due to protein other than S100B, the molecular identity of selected lanes was analyzed as shown in FIGS. 1A and 1B by MS.

The two antibodies gave comparable but not identical results. While both revealed a strong signal from brain tissue, muscle and fat also exhibited strong levels of expression. The data were quantified to construct the bar graph shown in FIG. 1C where the values are expressed in nanograms per lane. An added standard of S100B was used to quantify S100B expression in tissues. In addition, these expression levels were compared to brain tissue as shown in FIG. 1D. Note that when data are normalized to brain expression, significant levels were seen in extracranial tissues and the extent of this phenomenon depended on the antibody used.

### Molecular Identity of the S100B Signal

MS analysis confirmed that the brain tissue signal, consistent by molecular weight with S100B, was indeed this protein (Table 3). When the same lanes were isolated from fat samples, S100B expression was similarly found. Fat tissue was chosen because it was most consistently reported in the literature to contribute to elevated serum S100B levels [Anderson, R E, et al., Ann Thorac Surg, 71:1512-1517 (2001); Steiner, J., et al., Psychoneuroendocrinology, 35:321-324 (2010)]. In addition to S100B, MS analysis also revealed the presence of another protein of the S100 family, namely S100A1 [Donato, R., et al., Int J Biochem Cell Biol, 33:637-668 (2001)].

Results with a Clinically Relevant Platform

Most of the clinical results dealing with the utilization of S100B as a predictor of neurological disorders were obtained with one of several immunoassays that are commercially available (for a complete recent summary see Goncalves C A, et al., Cllin Biochem, 41:755-763 (2008)).

S100B was then measured by ELISA (Sangtec-LIAISON; FIG. 2C). With the LIAISON system, brain was the chief expressor, while other tissues contributed less significantly, if at all to the signal. In particular, neither muscle nor fat gave a significant signal as it did in the Western blots. Thus, tissue samples S100B content was substantially different when using ELISA vs. Western (compare FIG. 2C and FIG. 1C).

In addition to this commercially available immunoassay, previous literature dealing with clinical samples has often used an automated version of the same test [Goncalves C A, et al., Cllin Biochem, 41:755-763 (2008)]. The results of a direct comparison between the automated and manual test are shown in FIG. 2A. Note that a good correlation existed between the two tests. In addition, we compared the results of a clinically relevant test based on the Sangtec-Diasorin platform with another commonly employed platform (Roche Diagnostic, FIG. 2B). Again, a good correlation between the two systems was noted. These results have shown that there is a significant quantitative difference when comparing results obtained by gel-based analysis versus ELISA-type immunoassay systems. The latter, produced comparable results both quantitatively and qualitatively, indicating that the use of these clinically relevant platforms leads to meaningful and comparable results.

### Molecular Nature of S100B Released by Brain Tissue

The literature dealing with the detection of S100B as an indicator of neurological dysfunction or BBB leakage was profoundly affected by the discovery of Marchi et al. (Epilepsia, 48:732-742 (2007)), who demonstrated that upon iatrogenic BBB disruption (BBBD) S100B levels were elevated minutes after the procedure itself, which prompted characterization of S100B as an indicator of BBB damage rather than a protein related to neuronal cell death or other types of brain injuries. However, recent findings by others have shed doubt on the utility of this approach, primarily because of extracranial sources of S100B. The data herein, in fact, show that this is indeed the case and that tissue other than brain expresses this protein. However, it is also known that S100B may be detected in its monomeric or dimeric form. In addition, S100B may form a homo- or hetero-dimer with its companion S100A1.

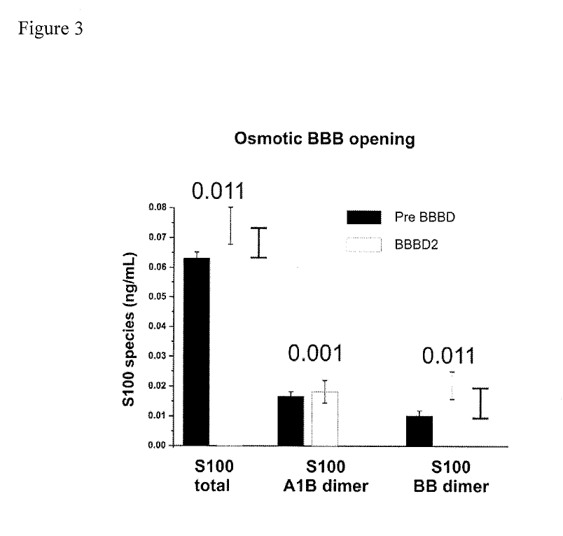

The molecular nature of the S100B extravasating from the human brain under conditions of iatrogenic BBB disruption was investigated (FIG. 3). To this end, ELISA platforms manufactured by Canag/Fujirebo were used to specifically dissect out the signal components due to homo- or hetero-dimers of S100B and S100A1. In FIG. 3, note that BBBD caused an increase of S100B dimer but not of the S100A1-B heterodimer. The increase of the B-B homodimer was the principal event that caused the elevation of the total signal. In other words, these results demonstrated that the main species of S100B released by the brain when the blood-brain barrier is disrupted is the S100 B-B. The numbers above the histogram refer to ng/mL changes occurred between pre-BBBD and at the time when chemotherapy was injected. Note that virtually all the increase in total S100B measured was due to the B-B dimer (0.011 ng/ml). The quantitative results for total S100 are similar to what published previously.

### Do Extracranial Sources Contribute to the Clinical Test for Serum S100B?

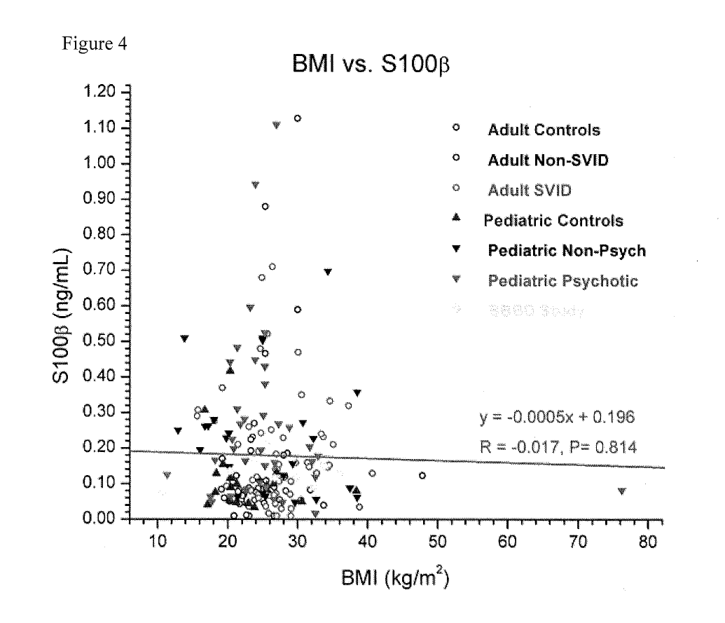

The results presented so far unveiled a complex scenario where several different molecular species act, upon BBBD, to modify serum values of S100B. Whether the S100B values in serum of patients or controls across the broadest published population of subjects was investigated. The results are summarized in FIG. 4 and Table 1. Given the expression of S100B in adipocytes, the relationship between fat content and S100B levels was investigated. This was achieved by determining body mass index (BMI; see Methods for calculations). Note that when serum samples from 200 subjects were analyzed, no correlation was found between BMI (or body weight in kg, not shown) and S100B levels (FIG. 4). This indicates that while unquestionably S100B is present in fat, it does not alter the S100B serum concentration in “normal” volunteers, patients affected by a variety of disorders, or pediatric controls or children with various illnesses. These results are also consistent with findings by others who evaluated the relevance of BMI in serum values of S100B in a large number of controls and traumatic brain injury patients.

### Discussion

Shown herein is that in spite of robust expression by extracranial sources, changes in serum levels are primarily dictated by extravasation across the disrupted BBB. In addition, the main molecular species of total S100B related to blood-brain barrier disruption is the S100B homodimer.

### Methodological Considerations

The study described herein compares and cross-validates various approaches to the detection of S100B. In addition, a vast array of subjects and tissues have been studied. Western blot analysis revealed that, regardless of the antibody used, S100B is found in tissues other than brain. The results show, surprisingly, a poor correlation between different antibodies. While cross-reactivity of antibodies is well known and widely accepted, it was nevertheless surprising that the rank order of expression depended on the antibodies used. In addition, testing by ELISA showed a different profile, where expression by extracranial sources was less prominent. However, the gel-based approach was sensitive for S100B which was detected by MS. The co-expression of S100A1 was expected, based on results by others. Although discussion of the possibilities of why such varying expressions of S100B protein between the platforms of Western blot and ELISA was observed is very noteworthy. These findings may have implications beyond recent S100B research efforts, inasmuch as most of the current knowledge on protein function is based on antibody detection of levels by gel electrophoresis. The use of antibody-independent detection therefore is advisable.

### Limitations and Strengths for Basic Scientists

Different levels of S100B expression using two different antibodies, and poor qualitative and quantitative correlation were found. Similar results were obtained in rodent tissue with other commercially available antibodies.

A BMI calculation was used to assess individuals' relative body fat. Recent studies on nutrition and metabolism have validated techniques such as ultrasound, air displacement plethysmography and bioelectrical impedance to be superior to BMI for accurately measuring body fat. These techniques were not readily available nor could they be easily implemented and therefore the BMI calculation was utilized not only out of practicality, but also out of the widespread use of it in other S100B studies.

### Clinical Significance

Demonstrated herein is that the clinical detection of S100B is more reproducible and robust. In addition, the type of instrument, or the platform used did not alter the results, nor did it affect the predictive value of the test. The clinical tests all measure total S100B, regardless of it monomeric or dimeric state, nor do they consider whether S100B is bound to S100B or S100A1. Preliminary results with tests detecting only S100B-S100B dimers have demonstrated, as expected, that the ceiling for “normal values” is significantly lower than the published “0.1 ng/ml” dogma [1], [6], [10], [14], [19], [24], [40], [44]-[46] (Kanner A. A., et al. (2003) Cancer 97: 2806-2813; Rothermundt M., et al. (2004) Int Rev Neurobiol 59: 445-470; Biberthaler P., et al. (2001) World J Surg 25: 93-97; Fazio V., et al. (2004) Ann Thorac Surg 78: 46-52; Anderson R. E., et al. (2001) Ann Thorac Surg 71: 1512-1517; Vogelbaum M. A., et al. (2005) Cancer 104: 817-824; Mussack T., et al. (2000) Acta Neurochir Suppl 76: 393-396; Anderson R. E., et al. (2001) Neurosurgery 48: 1255-1258; Jonsson H., et al. (2000) J Cardiothorac Vase Anesth 14: 698-701; Raabe A., et al. (2003) Restor Neurol Neurosci 21: 159-169).

Confirmed herein is that S100B was not exclusively produced by CNS cells. It was found that muscle and fat were chief extracranial expressors, which is consistent with the literature [Anderson R. E., et al. (2001) Ann Thorac Surg 71: 1512-1517]. However, the results are in sharp contrast with the findings linking serum S100B to BMI [Steiner J., et al. (2010) Psychoneuroendocrinology 35: 321-324]. These results used the same system used by us (LIAMAT), but their sample size was significantly smaller. In addition, no cross-validation with other detection systems was used. The range of BMI values was larger, the ages broader, and the racial samples were balanced to reflect the general US population. Diseased patients' samples were also included to add to the clinical significance of the findings. No correlation between S100B and weight or height was found, but, as expected, a correlation with age was found [Portela L. V., et al. (2002) Clin Chem 48: 950-952]. When the values were restricted within a given category (e.g., pediatric controls in Table 1), no correlation between BMI and S100B was found. Why these results are in sharp contrast with Steiner et al. [Steiner J., et al, (2010) Psychoneuroendocrinology 35: 321-324] remains at present unknown.

The results show that extracranial sources of S100B do not significantly affect serum levels. Thus, the reported low sensitivity and positive predictive value (relative to the reported strong specificity and negative predictive value) for S100B [Vogelbaum M. A., et al. (2005) Cancer 104: 817-824] is not apparently due to extracranial release of the protein.

There are two possible explanations that may account for the discrepancy between previous studies documenting elevated S100B levels from extracranial sources and the present findings. There have been recent reports that serum S100B levels are positively correlated with body mass index without evidence of traumatic brain injuries. Interestingly, obesity has been hypothesized to be a state of heightened systemic oxidative stress and inflammatory response, which is mechanistically linked to other co-morbid conditions such as hypertension and small vessel disease. Therefore, it is not clear whether obesity itself or obesity-associated comorbidities contribute to a rise in serum S100B in the previous studies. In this study, the tissue specific expression of S100B in addition to serum S100B was measured, which represents a collective source from multiple disease processes. The study showed that an increase in fat mass might not in isolation be a major contributor to elevated S100B levels. Rather, obesity-related diseases are likely contributors. For example, small vessel ischemic disease associated with obesity is a source of serum S100B [Vogelbaum M. A., et al. (2005) Cancer 104: 817-824; Mazzone P. J., et al. (2009) PLoS One 4: e7242].

Prior studies have shown that cardiothoracic surgeries resulted in higher serum levels of S100B. However, Fazio et al. demonstrated that S100B antibodies from certain ELISA kits might cross-react with other proteins found in serum [Fazio V., et al. (2004) Ann Thorac Surg 78: 46-52]. It is difficult to characterize biomarkers in serum because of the wide range of protein concentrations and predominance of 10 to 20 proteins (albumin, immunoglobulins, etc.) that overwhelm the less abundant signals. This indicates that in other studies yet unknown cross-reactants were artificially increasing the apparent S100B levels measured in serum.

### Example 2

S100B homodimer is expressed in human brain astrocytes. The use of the Proximity Ligation Assay™ (PLA) (Olink Bioscience, Sweden) revealed that S100BB, the homodimer of S100BB, is the astrocytic and brain form of S100B (see FIGS. 5A-5B). FIG. 5A shows the fundamentals of PLA technology. When the PLA probes are in close proximity (<40 nm), the DNA strands can interact through a subsequent addition of two other circle-forming DNA oligonucleotides. After joining of the two added oligonucleotides by enzymatic ligation, they are amplified via rolling circle amplification using a polymerase. After the amplification reaction, several-hundredfold replication of the DNA circle has occurred, and labeled complementary oligonucleotide probes highlight the product. The resulting high concentration of fluorescence in each single-molecule amplification product is easily visible as a distinct bright dot when viewed with a fluorescence microscope.

In the experiments shown in FIG. 5B, two identical antibodies were used. These were conjugated to plus and minus probes. There were no secondary antibodies for this experiment. SIGMA monoclonal antibody anti-S100B Prod. N. S2657 was sued. The dilution was 1:100. The photomicrograph depicts a section of human brain, roughly at the cortical layers 3-6. Note red S100BB signal in cell bodies and pericapillary space (astrocytic end feet). The astrocytes in FIG. 5B have a typical morphology, and in addition to a well differentiated parenchymal component also send end feet to engulf the blood vessel. ECNu (light DAPI staining) and PeNu (robust DAPI) refer to nuclei of an endothelial cell and a pericyte.

### Example 3

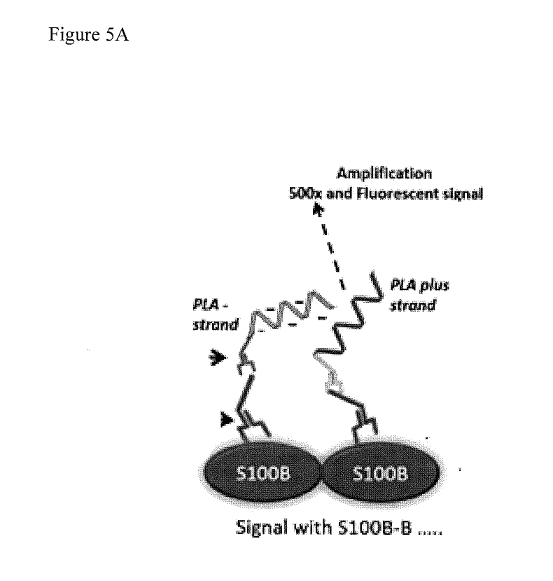

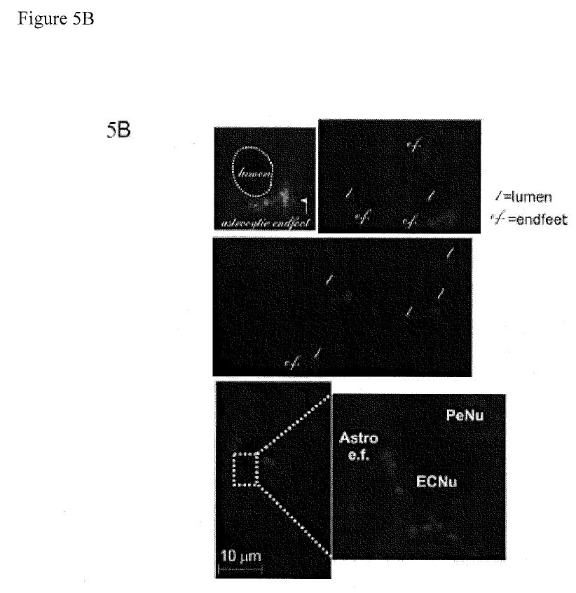

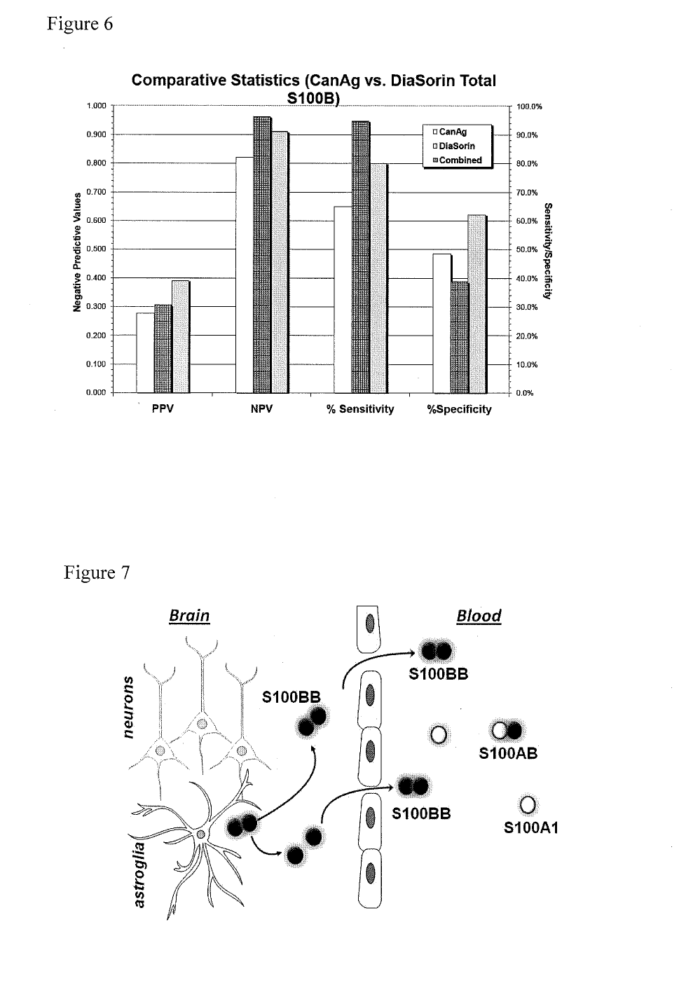

S100B tests used in combination have an improved negative predictive value and are thus less likely to reveal false negative patients. The test was used to analyze serum samples taken from patients diagnosed with systemic lung cancer (small cell and non-small cell carcinoma) who were at risk of brain metastases or with ongoing metastatic disease. The negative predictive value refers to contrast enhanced MRI scans used to visualize brain masses. NPV and PPV are negative and positive predictive value respectively. Specificity (negative predictive value) shows the proportion of negatives which are correctly identified. There was a synergistic benefit when S100B total by Diasorin and S100B Total by Canag in ruling out the presence of brain metastases diagnosed in this population by MRI. Blood was drawn at first diagnosis of systemic lung cancer; within a few days, the MRI scan was performed. Gadolinium was used as a contrast agent to reveal by MRI blood-brain barrier leakage due to tumor invasion in the brain. See FIG. 6.

### Example 4

### Efficacy of Anti-Inflammatory Therapy in a Model of Acute Seizures and in a Population of Pediatric Drug Resistant Epileptics

### Methods

### Rodents

Rats were housed in a controlled environment (21±1° C.; humidity 60%; lights on 08:00 AM-8:00 PM; food and water available ad libitum). Procedures involving animals and their care were conducted in conformity with the institutional guidelines that are in compliance with international laws and policies (EEC Council Directive 86/609, OJ L 358, 1, Dec. 12, 1987; Guide for the Care and Use of Laboratory Animals, U.S. National Research Council, 1996). Cleveland Clinic IACUC approved the protocol number 08491 for the performance of the presented experiments.

### Induction of Seizures and Drug Treatments

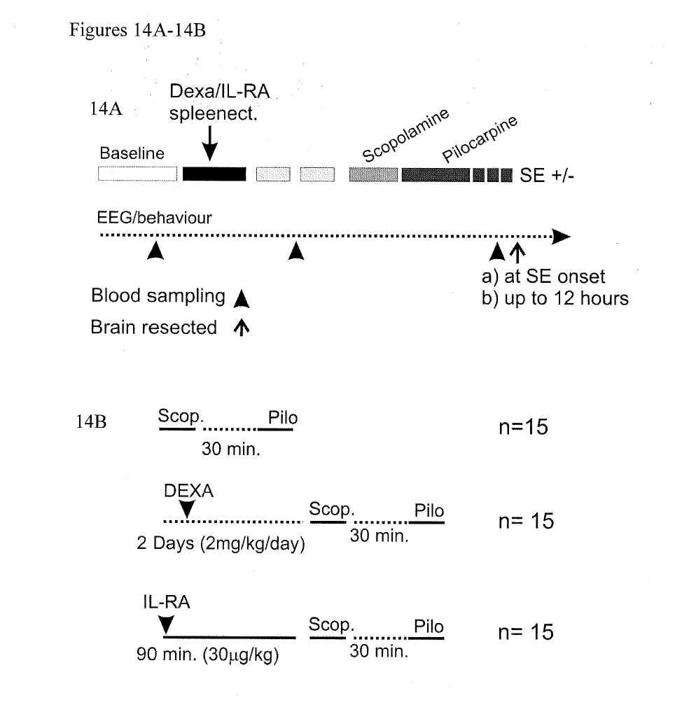

Rats (male Sprague-Dawley 225-250 g) were injected with methylscopolamine (0.5 mg/kg, i.p., Sigma-Aldrich) and 30 minutes after with pilocarpine (340 mg/Kg, Sigma-Aldrich). Data obtained from a total of 45 rats was analyzed (see also FIG. 14B). Development of seizure and status epilepticus was evaluated by behavioral (Racine's scale) and EEG assessment. Dexamethasone sodium phosphate (APP Pharmaceutical, IL, USA) was administered 2 mg/kg, i.p. twice a day for 2 days prior scopolamine/pilocarpine treatment. A single dosage of 1 mg/day was also used but did not exert any discernable effects (data not shown). IL-RA data included here are relative to [Marchi N., et al. (2009) Neurobiol Dis 33: 171-181]. IL-RA was administrated in the tail vein (30 μg/kg) 2 hours before scopolamine/pilocarpine treatment.

### Schedule of Treatment-Sampling and Sacrifice

The number of rats used, timing of drug treatments, blood drawings-analysis and animal sacrifice are indicated in FIGS. 14A-14B. Note that blood samples were taken at baseline, after each treatment and immediately before (or at onset) of status epilepticus (SE), as evaluated by EEG and behavioral (Racine's Scale) assessments. This methodology has been used in the past to evaluate the presence of BBB damage and inflammatory process preceding seizure onset [Uva L., et al. (2008) Neuroscience 151: 303-312]. Rats that developed SE despite dexamethasone pre-treatment were either sacrificed at SE onset to determine BBB damage and serological correlates or followed by EEG/behavioral analysis up to 12 hours (seizures were not stopped with barbiturate) to analyze seizures severity and mortality rate (see FIG. 14A).

### Rodent EEG Recording and Video Monitoring

Stereotactic electrode implantation was performed in rats under isofluorane anaesthesia, using the Kopf stereotactic frame. Approximately, half of the rats used were implanted. Four stainless steel screws (MX-0090-2, Small Parts Inc., Miami, Fla.) were placed bilaterally on the dura mater of the fronto-parietal cortex. A prefabricated Pinnacle pre-amplifier was connected to the screws. The system has three bio-potential channels—2 EEG and 1 EMG. Prefabricated head implants (Pinnacle Inc., USA) ensured accurate electrode positioning and reliable, robust contacts. Cable artifacts are eliminated by pre-amplification of the EEG and EMG waveforms at the animal's head. EEG data were sampled a rate of 200 Hz. All data are transferred via a USB connection to a PC. Rats were left unrestrained for 2 weeks to recovery from surgery before EEG recordings were performed. Each rat was kept in a separate cage under 12-hours dark-light cycles with free access to food and water. Origin Microcal 7.0 and Diadem (National Instruments) was used in conjunction to the acquisition system for data analysis (e.g., time joint frequency analysis).

### FACS and IL-1β ELISA

The schedule of blood sampling is illustrated in FIG. 14A. Blood was collected from the tail vein using a fixed catheter (Venisystem, Abbocath, 22 G, 300-500 μl/sample). For FACS analysis, 150 μL of whole blood were incubated with a combination of specific antibodies recognizing T-cell subpopulation (CD3+). In particular mouse anti-rat CD8a-FITC, CD3-R-PE and CD4-PE-Cy5 (BD-Pharmingen) were added (2 μL, 5 μL and 5 μL respectively) to blood samples. Blood samples were analyzed by the FACS-Core facility at the Cleveland Clinic. IL-1β ELISA-tests was purchased from Pierce Biotechnology Inc. and performed as described by the vendor.

### Blood-Brain Barrier Integrity Assessment

BBB status was assessed using two independent modalities: serum S100β and brain Evan's Blue extravasation [Marchi N., et al, (2009) Neurobiol Dis 33: 171-181; Kanner A. A., et al. (2003) Cancer 97: 2806-2813]. Blood samples were obtained from the tail vein and brains were collected after sacrifice (control, dexamethasone-treated or at onset of SE, n=5 rats/group). S100β: blood samples were collected at the times indicated in FIGS. 14A-14B. Blood samples were centrifuged at 1,200×g for 10 min, and the supernatant serum stored at −80° C. The S100β concentration was measured using the Sangtec 100 ELISA method (Diasorin, Stillwater, Minn., U.S.A.). Evan's Blue: the pattern of BBB leakage was evaluated by measuring fluorescent signal present in the brain. The fluorescent solution was prepared reconstituting 2 g of Evans Blue in 100 ml of phosphate buffered saline (0.1 M PBS). The solution was stirred at room temperature in the dark. The solution was infused in the left heart ventricle (2 ml/rat at rate of 1 ml/min). Presence of gross leakage in treated and control animals was evaluated by fluorescent in vivo imaging signaling (IVIS). Brains were placed in the IVIS chamber and background fluorescence was set as zero. Digitalized signals were created following a blue-to-red scale based on the regional distribution of fluorescent signal.

### Human Subjects

The study was conducted according to the Declaration of Helsinki Criteria and to the procedures for compassionate drug administration approved by the Ethic Committees of Carlo Besta Institute (Milan, Italy). Oral informed consent from was obtained from parents by specialized neurologists. Administration of GCs was considered compassion care since all patients had intractable life threatening seizures. The clinical data of 43 patients (Table 4) of the Carlo Besta Neurological Institute (Milan, Italy) was reviewed. All the patients had a known history of intractable seizures and antiepileptic treatment with conventional AEDs was administered (Table 4). Patients with a history of epileptic syndromes known to respond to steroids (i.e., West, Lennox-Gastaut, Landau-Kleffner and Rasmussen encephalitis) as well as patients affected by documented inflammatory brain disease were excluded. All the subjects received steroid treatment on an in-patient basis.

Different treatments were employed in different patients: ACTH, dexamethasone, methylprednisolone, and hydrocortisone. Details on glucocorticoids (GCs) or ATCH dosage are summarized in Table 4. Patients received GCs or ACTH therapy because of one of the following: 1) >50% increase in seizure frequency; 2) development of non-convulsive status epilepticus; 3) presence of epilepsia partialis continua (EPC). Before GCs or ATCH treatment, patients were evaluated by a team of neurologists, physiologists, and underwent EEG and routine laboratory examinations. Antiepileptic treatments were maintained during the steroid course (Table 4).

Steroidal treatment was, when successful, repeated in case of seizure recurrence. For this reason, the number of treatments reported is greater than the number of patients (treatments=92, see Table 4). GCs or ATCH therapy was considered successful when: 1) seizure frequency decreased by 50%; 2) status epilepticus was stopped; 3) or when epilepsia partialis continua (EPC) was stopped/reduced enough to allow voluntary movement in the affected body district. Duration of treatment was limited to acute dosing, which was repeated when the initial response was beneficial. The effects of length of treatments and efficacy/toxicity of chronic use are not presented herein, since this study design only addressed a proof of principle use of steroids in pediatric epilepsy.

### Statistical Analysis

Spike detection, spike area and instantaneous frequency calculation were performed using pClamp 9.2. Statistics were performed with aid of Origin 7.0 (Microcal) and Jump 7.0; data were considered to be significantly different when p<0.05 (by ANOVA or paired t-test for multiple comparisons). Normal distribution of data was evaluated with Wilk-Shapiro routine. Mosaic plots were graphed with Jump 7.0 and transferred to CorelDraw as metafiles. The Diadem (National Instruments) package was used to construct time-frequency plots. Fisher exact test was used (Jump 7.0) to evaluate the significance of probability of SE and incidence of mortality between groups of animals.

### Results

Described herein is the evaluation of the effect of anti-inflammatory agents in experimental seizures, and the efficacy of gluco-corticosteroids. The efficacy of anti-inflammatory treatments was evaluated in drug resistant pediatric epilepsies, excluding those conditions already known to benefit from steroidal treatment, i.e. West, Landau-Kleffner, Lennox-Gastaut syndromes and Rasmussen's encephalitis [Grosso S., et al. (2008) Epilepsy Res 81: 80-85; Sevilla-Castillo R. A., et al. (2009) Neuropediatrics 40: 265-268; Verhelst H., et al. (2005) Seizure 14: 412-421]. The response to gluco-corticosteroids, or ACTH was analyzed in a pediatric population and the results were used to develop a hypothesis that also takes into account data obtained from animal experiments where rats were exposed to convulsive doses of the cholinergic agonist pilocarpine.

The justification for extrapolating data obtained from pilocarpine-induced SE to drug resistant epilepsy may be considered inappropriate and one should ideally compare human data to pilocarpine-treated chronic rats who do not respond to AED. Thus, two points of asymmetry can be found in the current study, one related to chronicity of seizures in humans vs. acute nature of BBB disruption-induced seizures, as well as the issue of human epileptic vs. normal brain induced to seize. In fact, to segregate and study drug resistant rats would constitute the best animal correlate of human multiple drug resistance to antiepileptic drugs. However, recent experimental findings suggested that correlates of acute seizures (e.g., as triggered by iatrogenic BBB disruption or pilocarpine) are not dissimilar from chronic seizures. For instance, seizures acutely induced by intraarterial mannitol have EEG features similar to pilocarpine seizure and the histological and immunohistochemical tracts of acute seizures (e.g., cerebrovascular damage) are similar to the ones observed in the chronic epileptic human brain [Marchi N., et al. (2010) Blood-brain barrier damage, but not parenchymal white blood cells, is a hallmark of seizure activity. Brain Res.].

### Efficacy of Dexamethasone: Rat Study

Whether dexamethasone prevents the onset of pilocarpine-induced seizures in rats was evaluated, and seizure-induced mortality was quantified. Experimental details are shown in FIGS. 14A-14B. These effects are compared to those previously obtained using an IL-1 receptor antagonist [Marchi N., et al. (2009) Neurobiol Dis 33: 171-181].

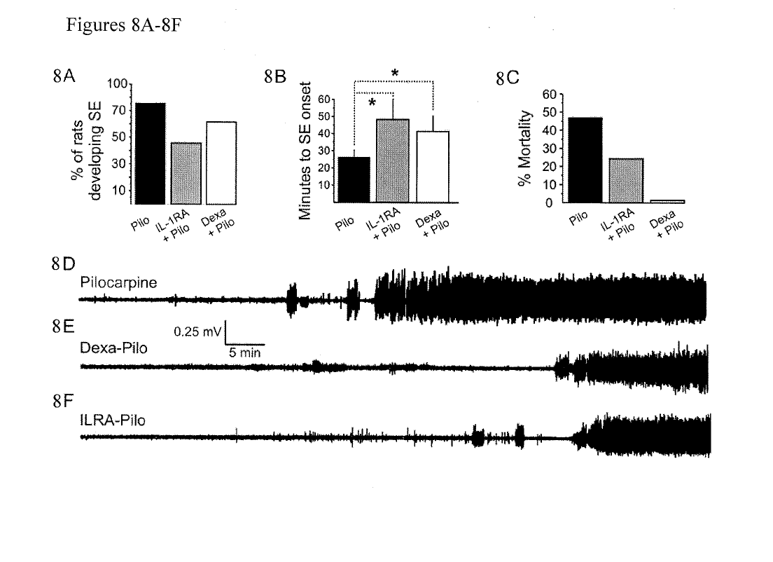

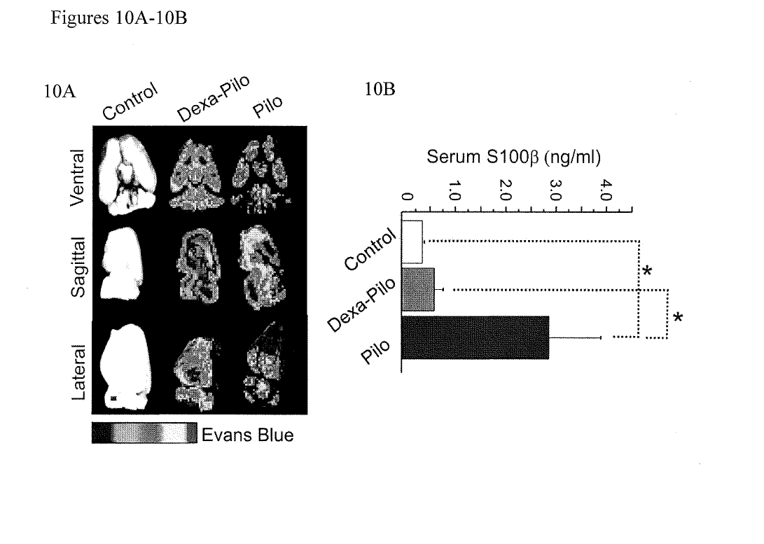

FIGS. 8A-8F shows typical EEG traces recorded after injection of pilocarpine alone (8D), after dexamethasone pretreatment (8E), or after blockade of IL-1β receptors (8F). The average results are shown in FIG. 8A-8C. Note that pilocarpine caused a stereotyped response consisting of an initial burst of action potentials followed by a full-blown and persistent SE. When anti-inflammatory treatment preceded exposure to the convulsive agent (see FIGS. 14A-14B), the number of rats developing develop seizures was reduced (FIG. 8A). When SE developed, the time of onset was significantly delayed compared to untreated controls (FIG. 10C). Mortality associated with SE was significantly decreased by dexamethasone (p=0.02, Fisher test).

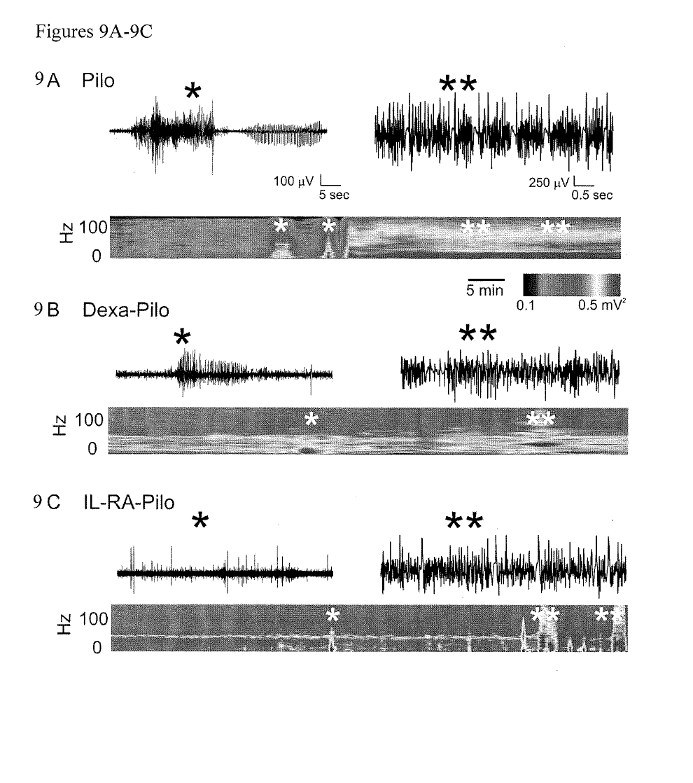

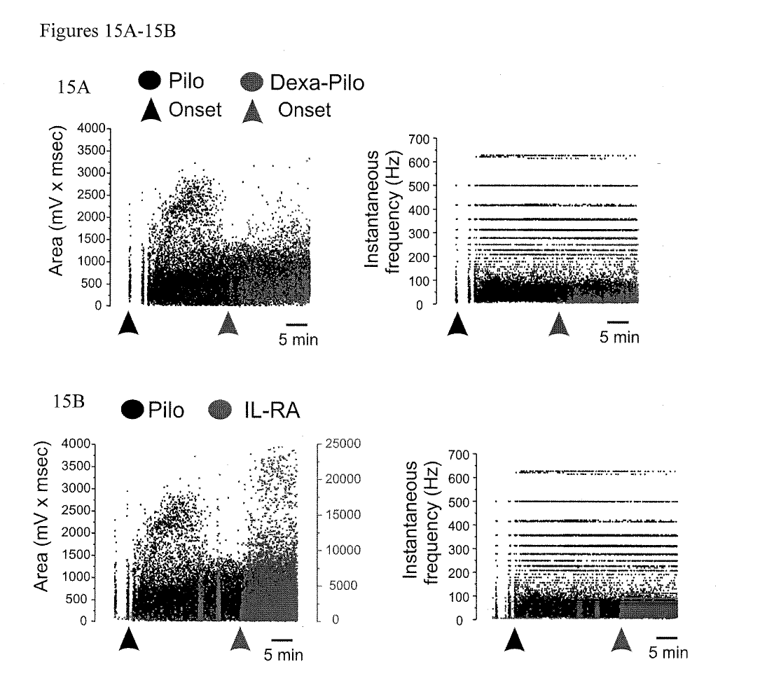

Time-joint frequency analysis was performed to examine changes not immediately apparent by EEG inspections. Note that the early burst clusters (single asterisks in FIGS. 9A-9C) were reduced in amplitude and frequency in animals pre-treated with either dexamethasone or IL1-RA. Severity of SE was also reduced in treated rats (frequency and amplitude distributions) as shown in FIGS. 15A-15B.

### Evaluation of Blood-Brain Barrier Damage

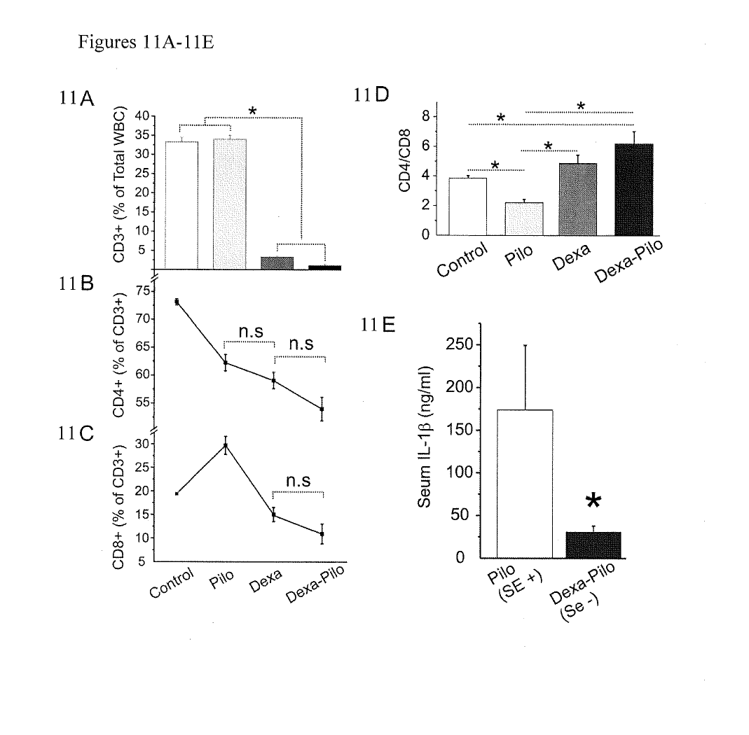

The integrity of the BBB in animals treated with pilocarpine and in those pre-treated with dexamethasone was compared (FIGS. 11A-11B). It has been previously demonstrated that pre-treatment with IL-RA prevents BBB damage and reduces seizure occurrence [Marchi N., et al. (2009) Neurobiol Dis 33: 171-181]. BBB integrity was evaluated by visualization of Evans blue extravasation and by measurements of serum levels of S100β [Kanner A. A., et al. (2003) Cancer 97: 2806-2813]. The significant increase of serum S100β levels and Evan's Blue signal measured at onset of SE was noted (FIGS. 10A-10B). Both S100β levels and Evan's Blue signal were comparable to control when seizures were prevented or reduced by dexamethasone. These results are in agreement with animal studies previously published [Ivens S., et al. (2007) Brain 130: 535-547; Hermsen C. C., et al. (1998) J Infect Dis 178: 1225-1227]. Remarkably, a decrease in MRI FLAIR hyperintensity concurrent with seizure reduction was observed in patients after corticosteroid treatment (see text below FIGS. 13A-13D).

### Serological Correlates of Dexamethasone Efficacy in Rats

Circulating white blood cells (WBCs) were analyzed to determine the level of T-lymphocyte activation after pilocarpine or after dexamethasone followed by pilocarpine. The most important effect of dexamethasone was a drastic reduction in the number of circulating T-cells (CD3+, FIG. 11A). Among the remaining CD3+ cells, the relative percentage of CD8+ subpopulation was significantly affected by dexamethasone (FIG. 11C). However, the latter change may be negligible owing the drastic reduction of total number of circulating T-cells (FIG. 11A). A change in CD4:CD8 ratio consistent with an inflammatory activation was seen after pilocarpine treatment (FIG. 11D, see also Marchi N., et al. (2007) Epilepsia 48: 1934-1946; Razani-Boroujerdi S., et al. (2008) J Neuroimmunol 194: 83-88). Dexamethasone significantly reduced the levels of serum IL1-β (FIG. 11E).

### Efficacy of Gluco-Corticosteroids or ACTH: Human Study

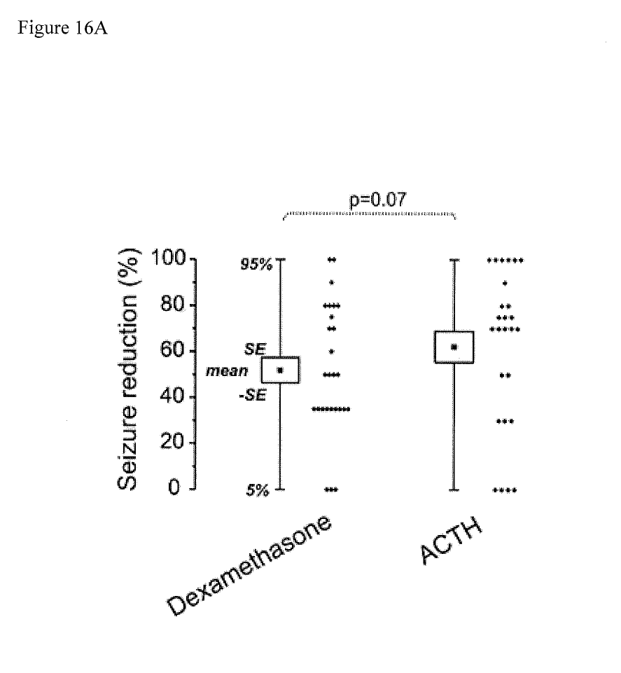

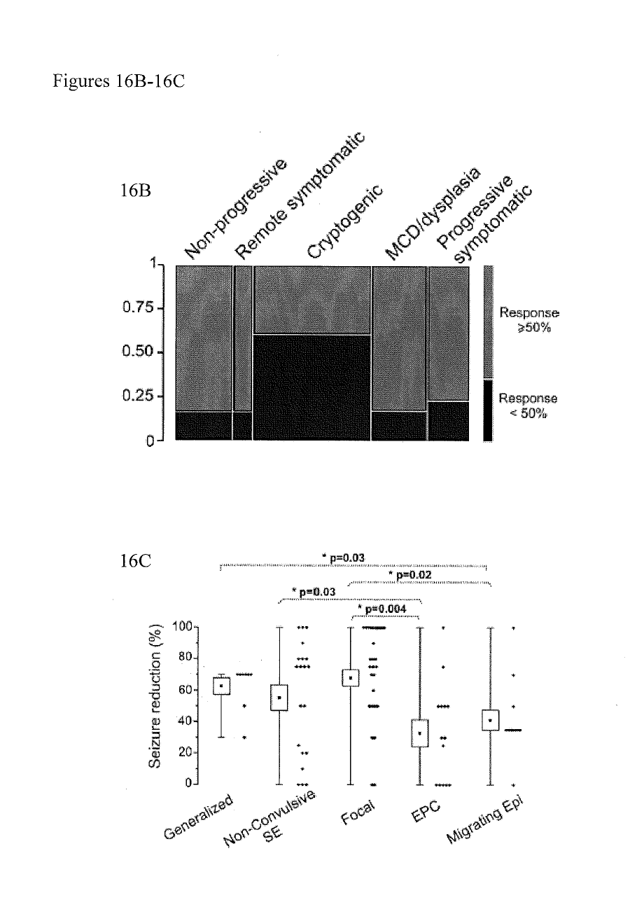

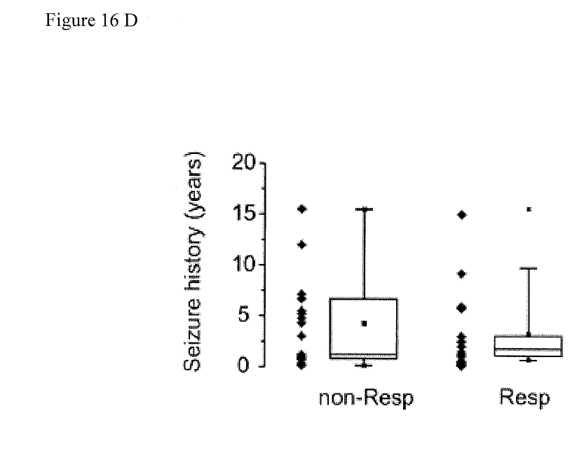

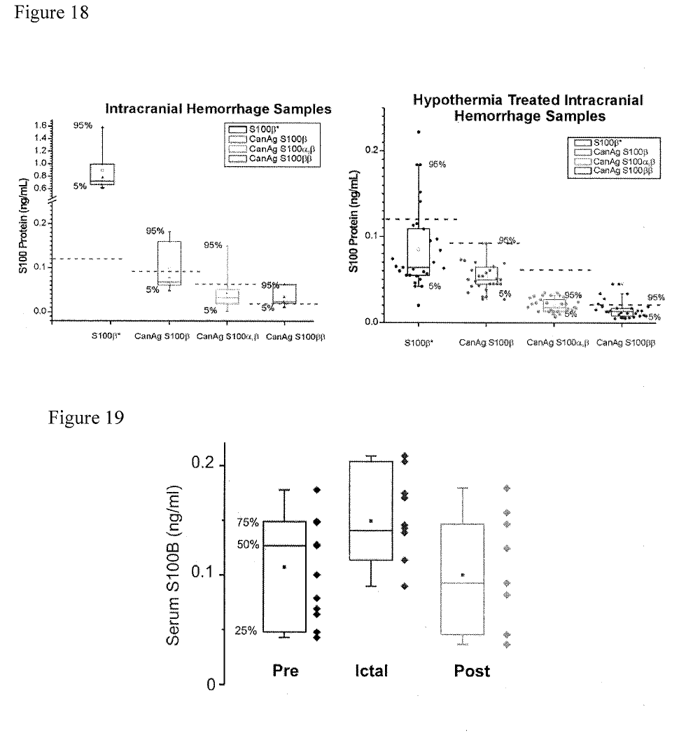

The efficacy of add-on glucocorticosteroids (GCs) or ACTH treatment in patients affected by drug-resistant epileptic seizures was investigated (see Table 4). Inclusion criteria are described in the Methods. For the whole study (FIGS. 16A-16D and 18) data from 43 patients (24 females and 19 males, see Methods) and a total of 92 GC treatments were reviewed. The data presented in FIGS. 13A-13B refer to 15 patients and 53 treatments with dexamethasone or ACTH. Mean age at GCs or ACTH treatment was 6.4±5.2 years. Etiology and type of epilepsy are summarized in Table S1. First seizure occurred between a few days after birth and 13 years of age (mean±SE: 2 years±8 months). Table 4 shows patients' AED regimens and, in bold, the AED co-administrated with GCs or ACTH.

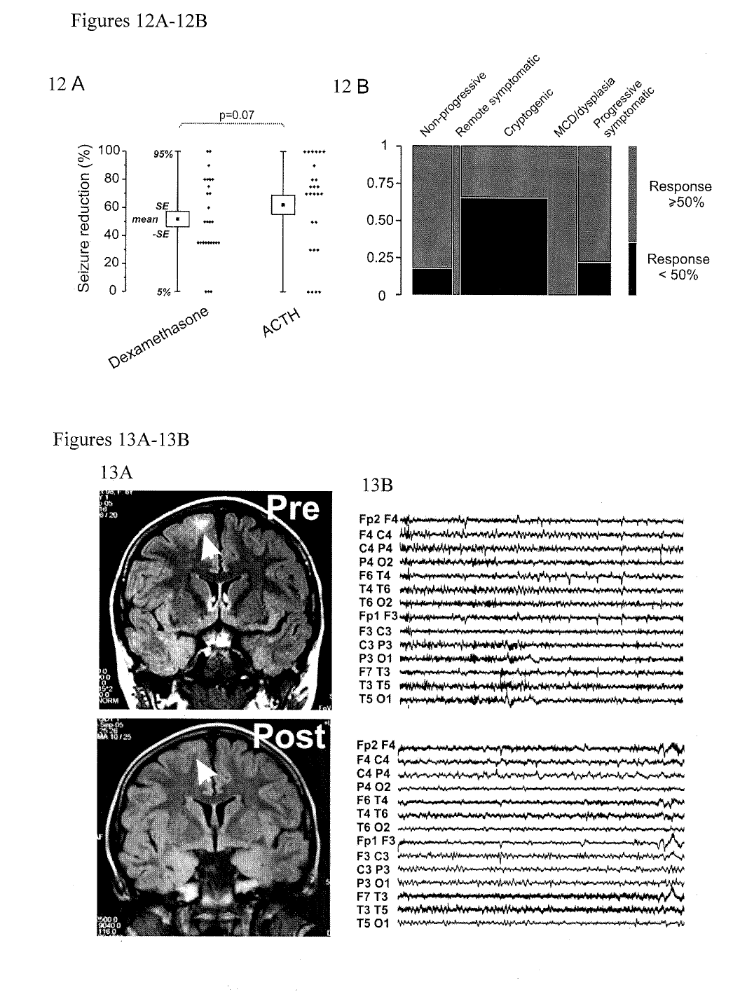

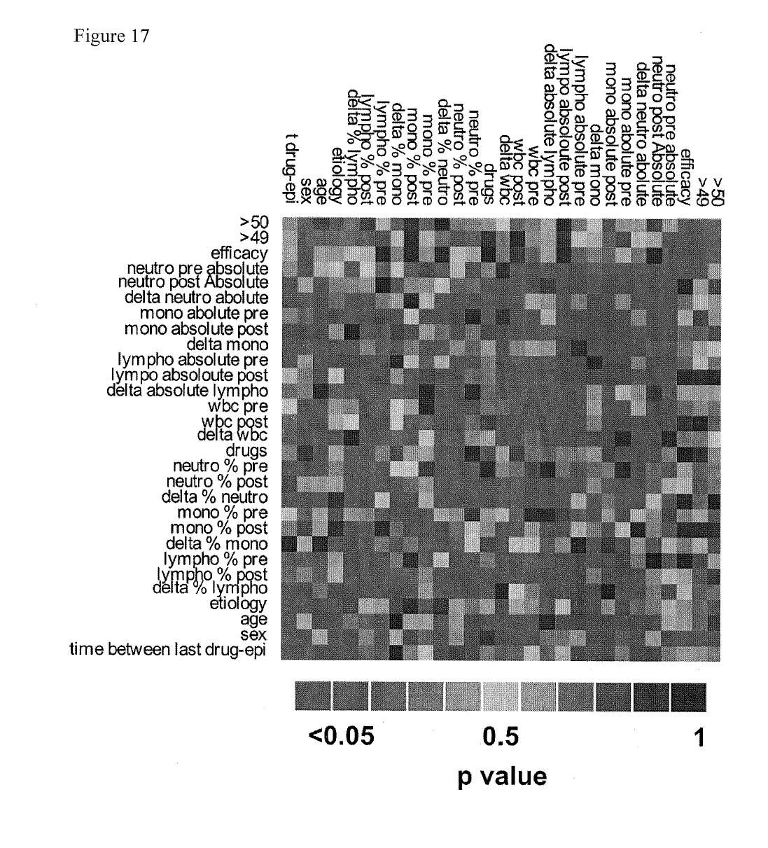

FIG. 12A shows the efficacy of dexamethasone or ACTH treatments. Overall, dexamethasone or ACTH significantly reduced occurrence of seizures. Overall, the response was variable ranging from complete reduction of seizures to no benefit from the treatment. The mosaic plot in FIG. 12B shows the distribution of the overall efficacies (set as ≧50%) across different etiologies. The overall results obtained with three different GCs (dexamethasone, methylprednisolone and hydrocortisone) are shown in FIGS. 16A-16D. The analysis of efficacy with regard to the type of epileptic syndrome shows that the best responders were patients with focal seizures (FIG. 16C). There was no correlation between seizure history (years) and response to treatment (FIG. 16D). The latter result is also shown in the multivariate analysis in FIG. 17. Common side effects associated with administration of GCs (e.g., increased body weight, anxiety and insomnia) were observed. Other side effects were only marginal and did not require cessation of therapy. Only in few cases (5%) GCs were suspended due to changes in coagulation, alteration of blood electrolytes or glycemia.

### Discussion

The results have shown that dexamethasone reduces the number of rats experiencing status epilepticus (SE) and abolishes mortality. The mechanism by which dexamethasone lessens pilocarpine seizure burden encompasses improved BBB function. This was shown by analysis of dye and marker extravasation in treated vs. untreated animals. The efficacy of add-on gluco-corticosteroids in a population of pediatric drug resistant patients excluding those syndromes known to be responsive to GCs and ACTH (L-G, L-K, West or Rasmussen's) was also studied. The effect was beneficial regardless of the pathology and epileptic syndrome. A selected case where a decrease in FLAIR signal was associated with seizure reduction was also observed. Previous studies have shown that FLAIR hyperintense regions or regions of gadolinium enhancement correspond to sites of BBB leakage [Cornford E. M. (1999) Adv Neurol 79: 845-862; van Vliet E. A., et al. (2007) Brain 130: 521-534; Amato C., et al. (2001) Eur J Radiol 38: 50-5; Huang C. C., et al. (1995) Eur Neurol 35: 199-205; Lansberg M. G., et al. (1999) Neurology 52: 1021-1027; Alvarez V., et al. (2010) Epilepsy Behav 17: 302-303; Ivens S., et al. (2010) J Neurol 257: 615-620].

### Anti-Inflammatory Therapy: a Human-Experimental Parallel

The findings are presented herein in a format where clinical data are presented together with animal results. This is appropriate because: 1) there is a recognized urgency to provide rapid therapeutic advancement by comparing clinical and laboratory results (Kwan P., et al. (2009) Definition of drug resistant epilepsy: Consensus proposal by the ad hoc Task Force of the ILAE Commission on Therapeutic Strategies. Epilepsia; Stefan H., Lopes et al. (2006) Acta Neurol Scand 113: 139-155; Elkassabany N. M., et al. (2008) J Neurosurg Anesthesiol 20: 45-4) the anecdotal use of corticosteroids in clinical epilepsy has recently expanded (see below), but its full potential for widespread use is limited by the lack of scientific validation of their use. The study described herein compared the efficacy of glucocorticosteroids and ATCH: 1) in patients across a wide spectrum of epileptic etiologies and syndromes, 2) with the exclusion of those syndromes known to be steroid responsive (i.e. L-G, L-K, West or Rasmussen's). The efficacy of steroids in patients were also compared with the effects observed in the pilocarpine model. It has been previously demonstrated that direct inhibition of leukocyte-mediated blood-brain barrier disruption, comparable to the effects of GCs, prevents SE in this model [Fabene P. F., et al. (2008) Nat Med 14: 1377-1383; Marchi N., et al. (2009) Neurobiol Dis 33: 171-181].

These patients failed at least three AED medications and were exposed to all traditional anti-epileptic care that benefits most of their drug-respondent counterparts. Thus, while use of a placebo arm is mandatory to evaluate once and for all whether anti-inflammatory therapy is a valid AED alternative, the studies herein have shown their utility at least as an add-on maneuver.

Animal data were obtained exclusively from adult rats; this does not appear to be a crucial limitation since patient age ranged from a few months to adolescence and no differences in GCs or ACTH effect were observed with regards to age. Finally, patients received a combination of GCs and anti-epileptic drugs (Table 4) while rodents did not.

### Non-Neuronal Mechanisms of Epilepsy: Role of the Blood-Brain Barrier

There are several lines of evidence suggesting that the blood-brain barrier could be a valid adjunctive target for anti-epileptic drug therapy. Animal studies that have shown that breaching the BBB is a reliable mean towards decreased seizure threshold [van Vliet E. A., et al. (2007) Brain 130: 521-534] or seizure development [Marchi N., et al. (2007) Epilepsia 48: 732-742; Seiffert E., et al. (2004) J Neurosci 24: 7829-7836]. More importantly, these animal studies have been supported by concurrent clinical data showing that blood-brain barrier disruption (BBBD) causes seizures in human subjects [Marchi N., et al, (2007) Epilepsia 48: 732-742] and that BBBD has a causative link with post-traumatic epilepsy [Korn A., et al. (2005) J Clin Neurophysiol 22: 1-9]. It has been previously demonstrated that prevention of BBB failure reduces seizure onset [Marchi N., et al. (2009) Neurobiol Dis 33: 171-181]. The data presented herein further support a pharmacological approach aimed to prevent BBB failure or restore BBB integrity.

There are important clinical correlations resulting from the findings. First and foremost is the fact that the efficacy of corticosteroids in pediatric epilepsy is not limited to epileptic encephalopathies, such as infantile spasms and Rasmussen encephalitis [Vezzani A., et al. (2005) Epilepsia 46: 1724-1743; Granata T. (2003) Rasmussen's syndrome. Neurol Sci 24: Suppl 4S239-S243; Granata T., et al. (2003) Neurology 60: 422-425]. Rather, the effects seemed to be pronounced in focal epilepsy, including those due to focal dysplastic lesions (FIG. 11A-11E and FIGS. 16A-16D). This is consistent with the results of recent studies suggesting the efficacy of corticosteroids in focal and generalized epilepsy [Grosso S., et al. (2008) Epilepsy Res 81: 80-85; Sevilla-Castillo R. A., et al. (2009) Neuropediatrics 40: 265-268; Verhelst H., et al. (2005) Seizure 14: 412-421] of different etiologies. The impact of corticosteroids on seizures during pre-surgical subdural grid EEG monitoring in drug-resistant children was recently demonstrated. A reduced seizure frequency was found in dexamethasone-treated patient compared with untreated [Araki T., et al. (2006) Epilepsia 47: 176-180].

The results of the experimental study herein support the hypothesis that inflammatory mechanisms and BBB damage contribute to seizure generation and severity [Ravizza T., et al, (2008) Neurobiol Dis 29: 142-160; Vezzani A., et al. (2010) Curr Opin Investig Drugs 11: 43-50]. The pattern of WBC activation was confirmed in the experimental model used [Marchi N., et al. (2009) Neurobiol Dis 33: 171-181]. Interestingly, in rats pre-treated with dexamethasone, a decrease in SE severity (FIGS. 8A-8F and 9A-9C) was observed at time of decreased number of circulating T-cells (FIGS. 11A-11E) and reduction of BBB damage (FIGS. 10A-10B).

The efficacy of corticosteroids observed in patients supports the hypothesis that seizures of different etiologies (including those due to congenital malformations or acquired brain damage) may be aggravated by inflammatory mechanisms and BBB disruption. The hypothesis linking BBB damage to seizures is in agreement with histological studies that have shown BBB dysfunction in human epileptic tissue and with MRI studies showing changes corresponding to the location of EEG activity in patients with partial status or focal epilepsy [van Vliet E. A., et al. (2007) Brain 130: 521-534; Amato C., et al, (2001) Eur J Radiol 38: 50-54; Lansberg M. G., et al. (1999) Neurology 52: 1021-1027; Paladin F., et al. (1998) Ital J Neurol Sci 19: 217-220; Paladin F., et al. (1999) Ital J Neurol Sci 20: 237-242; Tan K. M., et al. (2008) Epilepsy. Res 82: 190-193; Alvarez V., et al. (2010) Epilepsy Behav 17: 302-303; Vezzani A., et al. (2005) Epilepsia 46: 1724-1743]. The clinical study design herein did not include the systematic evaluation of MRI before and after treatments, nevertheless the decrease in FLAIR hyperintensity concurrent with seizure reduction after steroid treatment (FIGS. 13A-13B) indicates the hypothesis that restoring BBB integrity may be one of the mechanisms involved in the antiepileptic action of corticosteroids.

### Experimental Approximation of Clinical Epilepsy

Pilocarpine has been historically used to model human disease and several features of this model have common traits with human epilepsy [Leite J. P., et al. (1995) Epilepsy Res 20: 93-104; Leite J. P., et al. (2002) Epilepsy Res 50: 93-103]. For example, it was shown that pilocarpine seizures cause cerebrovascular changes which are consistent with those seen on MRI of patients [Leite J. P., et al. (2002) Epilepsy Res 50: 93-103]. These consist of homeostatic failure leading, in patients, to MRI hyperintensity and in animals to increased extravasation of intravascular (Evans blue) or cerebral-to-serum (S100β) indicators (FIGS. 10A-10B). In addition, pilocarpine induces seizures by an unexpected immunologic activation mediated by specific receptors and adhesion molecules [Fabene P. F., et al. (2008) Nat Med 14: 1377-1383; Marchi N., et al. (2009) Neurobiol Dis 33: 171-181]. These same molecules are the target of corticosteroids, and depletion of these adhesion molecules appears to protect against pilocarpine or clinical seizures.

If BBB leakage is the main initiator of seizures, and if BBB repair is protective against neurological disease, which are the mechanisms involved? The experimental evidence provided herein, together with previous findings [Vezzani A., et al. (2005) Epilepsia 46: 1724-1743; Marchi N., et al. (2007) Epilepsia 48: 1934-1946: Fabene P. F., et al. (2008) Nat Med 14: 1377-1383], point to an inflammatory-induced damage of the BBB. Corticosteroids have a profound effect on human and rodent BBB permeability [Vezzani A., et al. (2005) Epilepsia 46: 1724-1743; Hermsen C. C., et al. (1998) J Infect Dis 178: 1225-1227; Betz A. L., et al. (1990) Stroke 21: 1199-1204; Cucullo L., et al. (2004) Brain Res 997: 147-151; Gutin P. H. (1975) Semin Oncol 2: 49-56; Rook G. A. (1999) Baillieres Best Pract Res Clin Endocrinol Metab 13: 567-581]. Moreover, corticosteroids have an impact on the number of circulating T-cells. Downstream signaling by IL1-β appears to be a common thread in pilocarpine-induced seizures [Vezzani A., et al. (2005) Epilepsia 46: 1724-1743; Marchi N., et al. (2009) Neurobiol Dis 33: 171-181; Riazi K., et al. (2010) Epilepsy Res 89: 34-42]. It therefore seems reasonable to assume that the immune system acts in concert to produce BBB leakage and seizures, while the counteraction of corticosteroids has the opposite effect [Riazi K., et al. (2010) Epilepsy Res 89: 34-42]. These results further indicate that seizures and inflammation belong to the same chapter of neuro-immunology, as also shown recently [Maroso M., et al. (2010) Nat Med 16: 413-419].

An important correlate of the findings herein is the fact that animal data strongly indicate that anti-inflammatory treatments have a pronounced effect on survival, which in the pilocarpine model is usually achieved only by stopping SE with diazepam or barbiturates [Blair R. E., et al. (2009) Neurosci Left 453: 233-237]. This likely bears significant relevance for the treatment of catastrophic epilepsies in pediatric or adult settings.

It is important to note that a non-inflammatory mechanism for steroidal action exists, namely the modulation of GABA receptors [Rogawski M. A., et al. (2002) Int Rev Neurobiol 49: 199-219]. This is a possible interpretation of results but is unlikely given that a number of corticosteroids, ACTH, or IL1-RA exerted the same effects. While the results cannot fully confirm or rule out a specific neuronal action of steroids, the fact that the response spanned across a wide range of anti-inflammatory molecules makes this unlikely.

Finally, while steroidal treatment showed promise in the study described herein, the fact remains that the use of potent and potentially harmful anti-inflammatory drugs is not a viable long-term option. The treatment, when successful, had to be repeated once the efficacy waned. This is likely due to traditional, concurrent etiologic mechanisms including cortical dysplasia, other brain lesions, etc. The proposed scenario thus indicates that: 1) reduced BBB function on an abnormal cortical/hippocampal background facilitates seizures; 2) the lesional tissue itself promotes BBB dysfunction, cooperating towards a further decrease of the threshold for seizures; 3) steroids “repair” the BBB while having no impact on circuital and structural abnormalities. This reduces seizure probability; and 4) the anti-inflammatory efficacy decreases over time. This vicious cycle, to be interrupted requires simultaneous targeting of neurons and endothelial cells. In multiple drug resistant patients, AEDs are obviously not sufficient to reduce hypersynchronous firing: a new therapy combing anti-inflammatory potency with neuronal targeting may be the necessary and winning combination.

### Conclusions

The results herein indicate the potential application of GCs in treating drug resistant seizures and support further studies assessing the effect of GCs in experimental chronic seizures.

### Example 5

### Hypothermia

The experiment described herein measured S100B (total, S100AB, S100BB by Canag ELISA after subarachnoid hemorrhage (SAH). The antibodies used were Canag Prod. No. 708-10 (total S100B), 706-10 (heterodimer) and 701-10 (homodimer). In addition, total S100B was measured by an independent method (Diasorin, shown as S100B*). The data in FIG. 18 describe the effects of hypothermia on serum levels of S100B and its dimeric forms. As shown in FIG. 18, after a brain hemorrhagic event (left top panel), serum S100B (monomeric, heterodimeric, homodimeric) levels were increased above the respective thresholds (indicated by dotted lines). S100B (monomeric, heterodimeric, homodimeric form) levels were below thresholds in patients who were treated with hypothermia (right top panel). Successful hypothermia treatments were accompanied by a decrease in serum total S100BB.

Hypothermia is used to reduce blood-brain barrier damage (and development of brain damage) in patients suffering from an ischemic-hemorrhagic stroke. Note that regardless of the test used, there was a significant difference between hypothermia (HT) or normothermia (NT) patients. Hypothermia was induced using surface cooling in patients with severe acute ischemic stroke (part of a multicenter study “Cooling for Acute Ischemic Brain Damage, COOL AID I”). Patients were cooled for periods 12-72 hours, with a mean duration of hypothermia of 47.4±20.4 hours (including a rewarming period of 22.6±15.6 hours). Blood samples were drawn at 72 hours post-procedure. Serum was separated by centrifugation (2000 RPM, 10′) and stored at −80 C. Samples were then analyzed by ELISA and processed as recommended by the vendor.

### Example 6

### Seizures

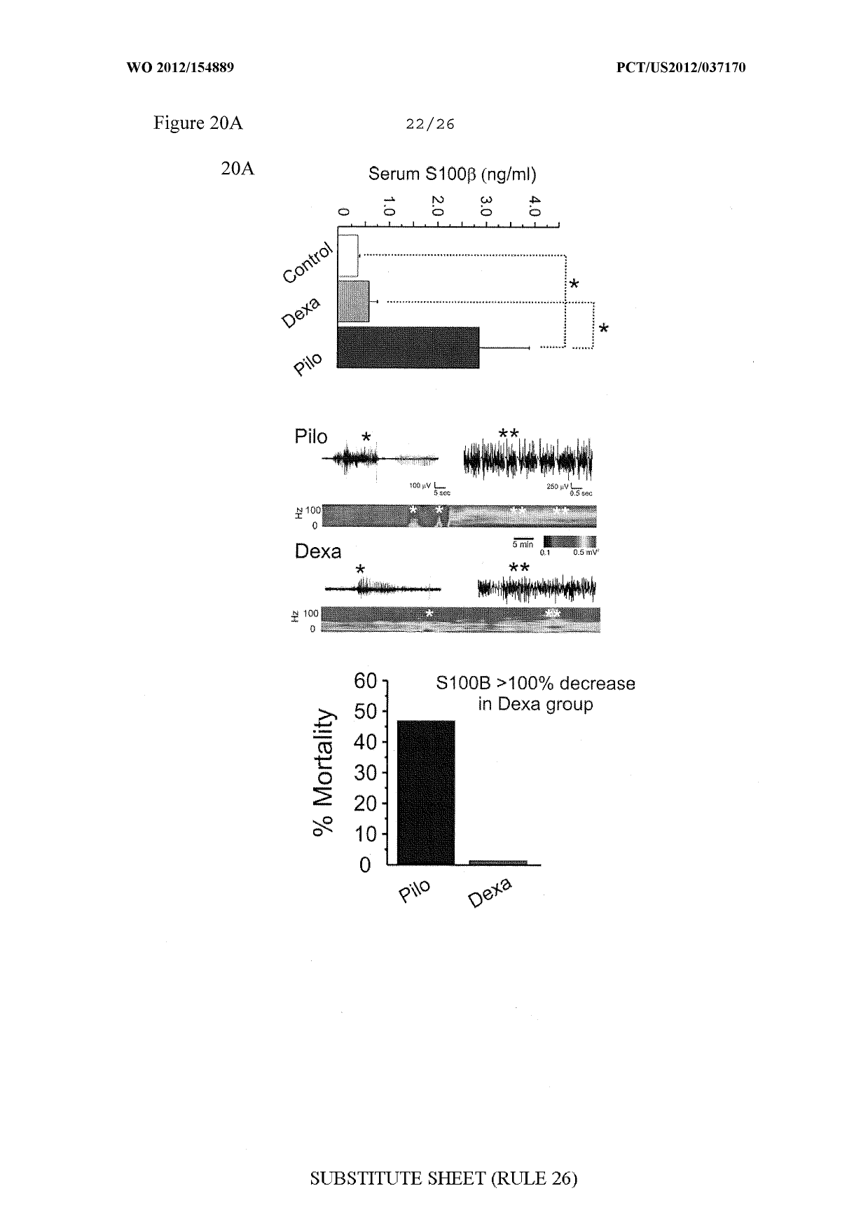

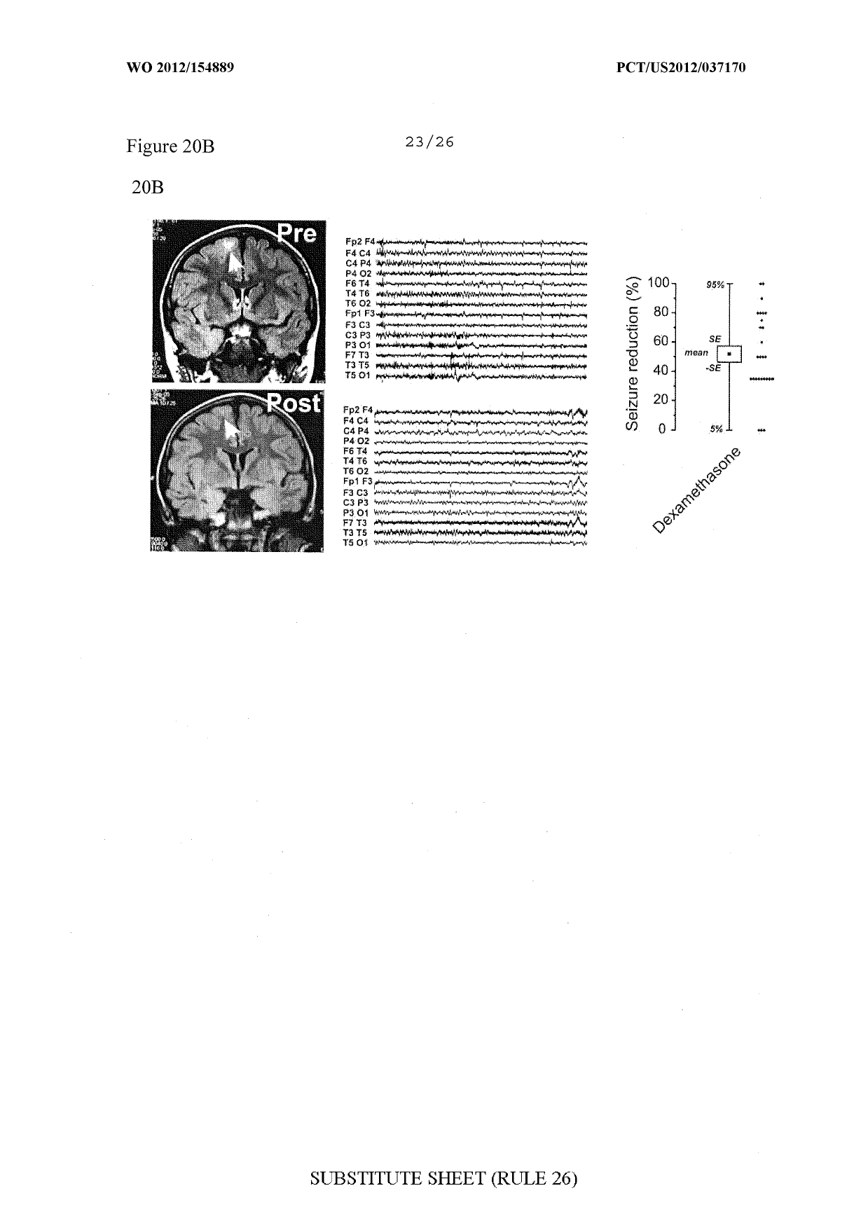

FIG. 19 shows yet another example on the use of S100B (total S100B, Diasorin) to monitor BBB permeability It is accepted that blood-brain barrier disruption represents a pathophysiological trigger of seizures 2. The data in FIG. 19 show the pattern of serum S100B increase in relation to seizure occurrence. Patients were monitored by EEG at Cleveland Clinic to determine seizure type and frequency. Patients' blood was collected pre-ictally and at time of detection spontaneous seizure as evaluated by EEG monitoring. Finally, a post-ictal sample was obtained within 24 hours from seizure activity. S100B serum levels were elevated immediately before and during an ictal event. S100B serum levels returned to baseline values during the post-ictal stage. Elevation of S100B levels in serum is a marker of the pathophysiological process (cerebrovascular failure) underlying seizure activity. Therefore, S100B measurement could be performed in conjunction to anti-seizure drug therapy. If the seizures are due to blood-brain barrier disruption, and associated therefore with increased marker levels, then a drug or maneuver that improves BBB function is recommended (see also FIG. 20A). Conversely, if a seizure is not causally associated with blood-brain barrier disruption then use of drugs targeting the BBB may be futile.

### Example 7

### Seizures

FIG. 20A show yet another example on the use of S100B (Total S100B Canag) as a serum marker of BBB integrity related to seizure propensity. Rats were treated with pilocarpine which is a seizure promoting agent. Note that the corticosteroid dexamethasone protected against not only propensity to seize but also reduced mortality. These effects on animals' wellbeing were accurately predicted by S100B (Total S100B Diasorin). Rats (male Sprague-Dawley 225-250 g) were injected with methylscopolamine (0.5 mg/kg, i.p., Sigma-Aldrich) as per protocol. Scopolamine is used to decrease the systemic effects of a cholinergic agent. 30 minutes after scopolamine the rats were injected with pilocarpine (340 mg/Kg, Sigma-Aldrich). Data obtained from a total of 45 rats were analyzed. Development of seizure and status epilepticus was evaluated by EEG assessment. Dexamethasone sodium phosphate (APP Pharmaceutical, IL, USA) was administered 2 mg/kg, i.p. twice a day for 2 days prior scopolamine/pilocarpine treatment. A single dosage of 1 mg/day was also used but did not exert any discernable effects. Blood samples were taken at baseline, after each treatment and immediately before (or at onset) of status epilepticus (SE), as evaluated by EEG and behavioral (Racine's Scale) assessments. This methodology has been used to evaluate the presence of BBB damage and inflammatory process preceding seizure onset. Rats that developed SE despite dexamethasone pre-treatment were either sacrificed at SE onset to determine BBB damage and serological correlates or followed by EEG analysis to analyze seizures severity and mortality rate.

As shown in FIG. 20A, S100B blood levels decreased in an animal model of epilepsy where restoration of BBB (as obtained by anti-inflammatory drugs) led to seizure reduction and to a long-term improved outcome (e.g., decrease mortality). As shown in FIG. 20B, improved blood-brain barrier function and decrease of seizure frequency was also observed in epileptic patients receiving anti-inflammatory therapy in this case dexamethasone at clinical dosage. Changes in BBB function can be monitored by T2-FLAIR MRI. Note the disappearance of sub-cortical hyperintensity in the post-treatment stage. The latter was associated with a significant long-term disappearance of sign and symptoms and improved outcome.

### Example 8

### Complicating Births

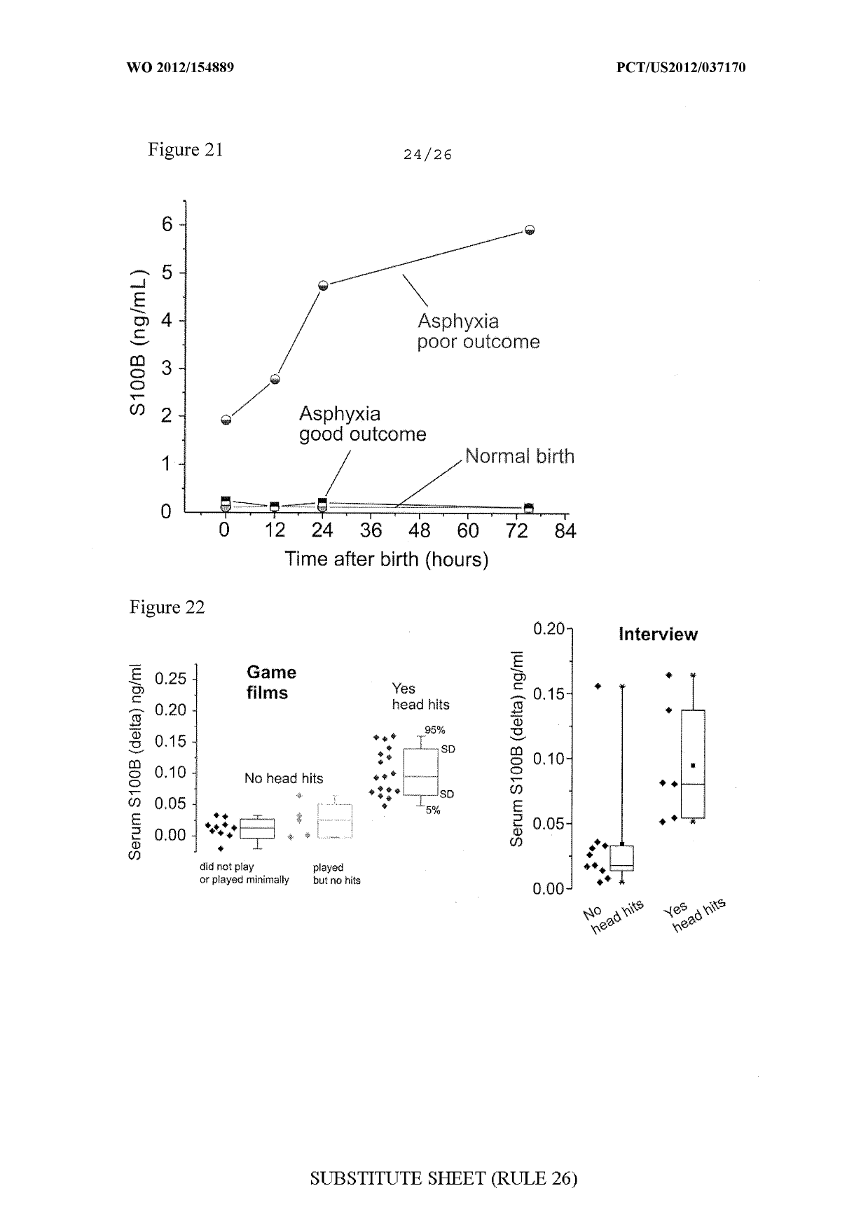

FIG. 21 shows how outcome of a birthing process with complications such as asphyxia can be monitored by using markers of BBB in body fluids. In this case, newborns were divided in three groups based on standard assessment methods. Normal birth without asphyxia, or asphyxia; the asphyxia group was further divided in asphyxia with poor or good outcome 48 hours after birth. Three patients per group are shown (mean values). In this example, markers of blood-brain barrier disruption were measured in urine since S100B is excreted by the kidney due to its low molecular weight. Thus, urine can be used as a surrogate for blood. Note that urine levels of S100B correlate with the time course of children progression after normal or asphyxia complicated birth. For these experiments we measured total S100B by Diasorin ELISA.

### Example 9

### Head Trauma

FIG. 22 shows how serum S100B (Total S100B, Diasorin) can be used to monitor the extent of potential head trauma in a population of college football players. Blood samples were collected from the players who consented to participate to the study. Blood was drawn the day before the game and within 1 hour after a game. Serum was separated by centrifugation (2000 RPM) and stored at −80 C. Elisa was performed as indicated by the vendor (Diasorin). Note that playing or not the game was not an essential factor in controlling S100B levels; thus, exertion alone does not cause changes in S100B. However, players who reported (by interview) or displayed by game tape analysis a number of severe head hits had elevated serum levels of S100B. These data show that even the slightest form of concussion (“ding” injury 3; 4) results in elevation of serum S100B levels. At baseline levels (before season) we found a significant difference between Caucasian and African-American players, the latter displaying higher values for yet unknown reasons.

### Example 10

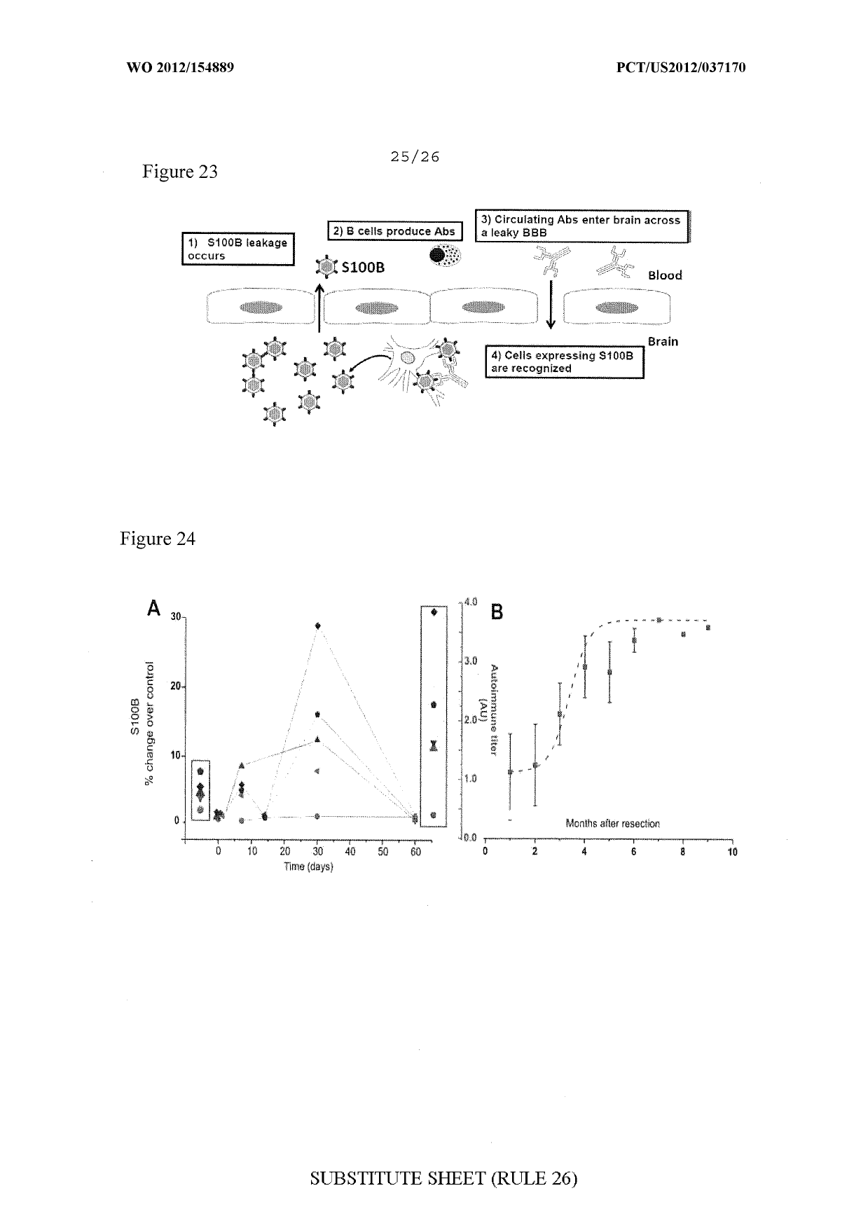

In this example, it is shown that anti-S100B auto-antibodies can be detected in serum of patients with a history of blood-brain barrier disruption. Results in FIG. 24 were obtained by measurements of anti-S100B auto-antibodies (AS100BAb) in serum of patients with diseases characterized by various levels of BBBD; the latter was measured by S100B total Diasorin and confirmed radiologically by gadolinium enhanced MRI. An ELISA system verified against another means of autoimmune detection (Western blot) was developed and a positive correlation was found between AS100BAb and serum S100B only in patients with S100B above cutoff, an indication of BBB damage. In other words, patients with no BBBD or no history of BBBD had low titers of A100BAb. Thus, autoantibodies are due to presentation of CNS antigen in diseases with a compromised BBB. To test the hypothesis that CNS unmasking by BBBD was involved, patients undergoing MR-guided laser-induced interstitial thermotherapy of recurrent glioblastoma were enrolled (FIG. 23 left panel). These patients were chosen because of the long MRI follow up periods (several months) and expected spikes in S100B correlating with MRI enhancement. In addition, the presence of tumor for prolonged times is associated with a sustained elevation of serum S100B (or other CNS markers). AS100BAb were elevated only after repeated spikes in S100B were recorded. In other words, the levels of autoimmune signal were elevated in patients with the highest cumulative S100B levels. The correlation between S100B and autoimmune titer was significant (p=0.02).

To test the hypothesis that increased serum S100B due to BBBD are sufficient to trigger an autoimmune response, AS100BAb in patients undergoing periodic, repeated, osmotic BBBD was measured. In these patients, and in analogous animal models, proportionality exists between serum S100B and extent of BBB disruption. Data show that BBBD triggers a time-dependent increase in autoimmune load and that the process itself peaks at approximately 6-8 months.

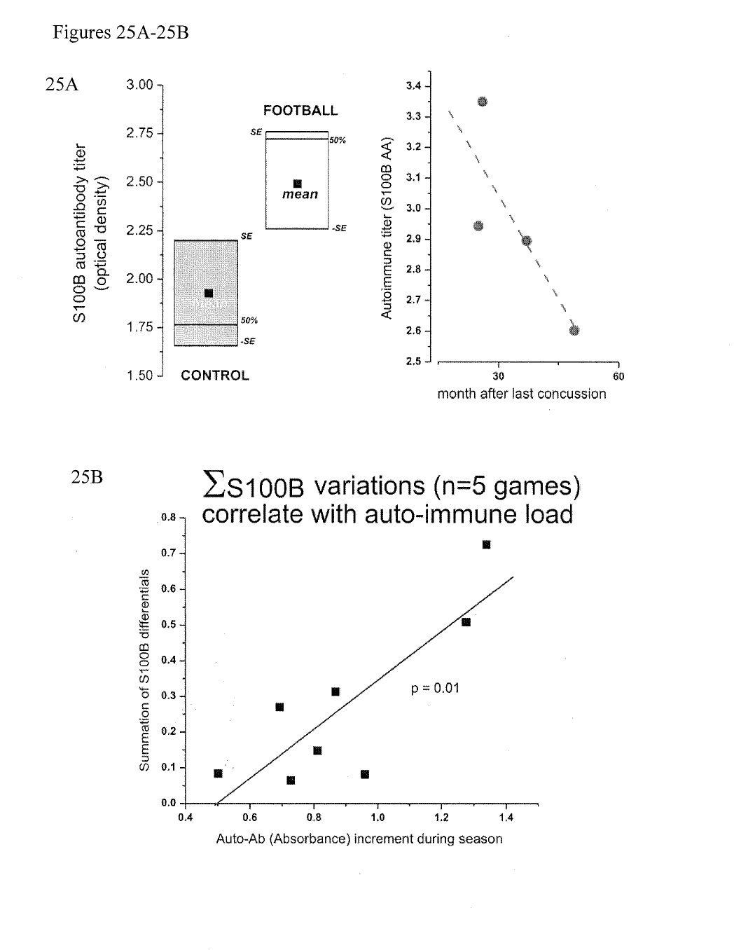

The time of autoantibody persistence after a minor traumatic brain injury was also measured. The data in FIG. 25A show that extinction of the autoimmune signal requires months.

The significance of these finding is that measuring autoantibodies against S100B is a tool to quantify or detect post-traumatic events.

The data in FIG. 25B show a linear and statistically significant relationship between appearance of S100B auto-antibodies (auto-Ab) and S100B elevations in blood. Data are relative to a Varsity football season. S100B auto-antibodies were measured in serum of players at the beginning and at the end of the season. Each data point in the graph refers to a specific player (n=8). X axis in FIG. 25B indicates players' S100B auto-Ab differential value: end of season−preseason, Y axis refers to the summation of serum S100B variations measured in players throughout the season. Note that players who experience the most frequent elevation in serum S100B also showed the highest titers of auto-Ab at the end of the season. Elevation of serum auto-AB against brain protein is associated with increased risk for cognitive decline. These data underscore the prognostic value of S100B/Auto-Ab measurements.

Provided below is an examples of methods that can be employed to detect autoantibodies against S100B in a sample such as human serum

1) Coated wells with 100 μL of human S-100β protein Catalog number-559291, EMD chemicals (1 μg/ml). Incubated overnight at 4° C.

2) Washed 3× with PBS.

3) Added 100 μL/well of 1% BSA as blocking solution; incubate at RT (room temperature) for 2 hrs

4) Aspirated & washed wells 3× with 200 μL of PBS containing 0.05% Tween 20/wash

5) Added 100 μL of serially diluted standards (positive ctrls), or serum samples (1:1000 and 1:5000) from patients to the 96 well Nunc Maxisorp plate. Incubate 1 hr at RT.

6) Aspirated & washed wells 3× with 200 μL of PBS containing 0.05% Tween 20/wash.

7) Added 200 μL of HRP (horseradish-peroxidase) Goat Anti-mouse IgG to positive controls and 200 μL of HRP conj. Goat anti-human IgG for serum samples. Incubated for 1 hr. at RT.

8) Aspirated & washed the wells, 3× with 200 μL of PBS containing 0.05% Tween 20/wash

9) Added 100 μL of OPD solution and incubate for 20-30 minutes at RT

10) Stopped the reaction by adding 100 μL of 2.5 M Sulfuric acid and read the plate using an ELISA plate reader @ 490 mu.

| | | | | | Height | | Weight | | BMI | | S100B | | | African | |
|---|---|---|---|---|---|---|---|---|---|---|---|---|---|---|---|
| Group- | | | Age | Height | Range | Weight | Range | BMI | Range | S100B | Range | % Fe- | Cau- | Amer- | Oth- |
| ing | N | Age | Range | (cm) | (cm) | (kg) | (kg) | (kg/m²) | (kg/m²) | (ng/mL) | (ng/mL) | male | casian | ican | er |
| Ped. | 20 | 16.6 | 14-18 | 171.3 | 161-191 | 67.3 | 45-118 | 22.9 | 16.7-38.4 | 0.11 | 0.03-0.42 | 55.0 | 14 | 1 | 5 |
| Con- | | (1.1) | | (7.3) | | (16.9) | | (5.1) | | (0.09) | | | | | |
| trols | | | | | | | | | | | | | | | |
| Ped. | 25 | 14.3 | 8-18 | 156.9 | 120-185 | 65.6 | 21-111 | 25.3 | 12.9-38.5 | 0.23 | 0.05-0.70 | 40.0 | 19 | 1 | 5 |
| (Non- | | (3.3) | | (18.9) | | (28.9) | | (7.7) | | (0.17) | | | | | |
| Psych) | | | | | | | | | | | | | | | |
| Ped. | 39 | 14.3 | 10-18 | 162.6 | 137-186 | 68.3 | 27-205 | 25.4 | 11.3-76.2 | 0.26 | 0.02-1.11 | 43.6 | 18 | 16 | 5 |
| Psy- | | (2.6) | | (13.4) | | (29.0) | | (9.5) | | (0.23) | | | | | |
| chotic | | | | | | | | | | | | | | | |
| Adult | 25 | 39.1 | 22-60 | 169.1 | 147-183 | 68.1 | 50-82 | 23.8 | 19.1-31.8 | 0.08 | 0.04-0.27 | 40.0 | 13 | 1 | 11 |
| Con- | | (11.1) | | (8.1) | | (9.3) | | (2.8) | | (0.05) | | | | | |
| trols | | | | | | | | | | | | | | | |
| Adult | 22 | 62.5 | 47-80 | 171.1 | 155-189 | 79.1 | 54-127 | 26.1 | 19.3-47.8 | 0.22 | 0.01-0.13 | 27.3 | 20 | 2 | 0 |
| (Non- | | (9.5) | | (10.1) | | (18.4) | | (6.1) | | (0.29) | | | | | |
| SVID) | | | | | | | | | | | | | | | |
| Adult | 59 | 65.8 | 49-85 | 169.6 | 146-203 | 78.8 | 37-125 | 27.3 | 15.6-40.7 | 0.18 | 0.01-0.71 | 40.7 | 53 | 6 | 0 |
| (SVID) | | (8.0) | | (10.5) | | (16.7) | | (5.1) | | (0.15) | | | | | |
| BBBD | 10 | 53.6 | 21-72 | 165.3 | 152-178 | 73.8 | 29-105 | 26.8 | 10.6-36.0 | 0.14 | 0.07-0.22 | 80.0 | 10 | 0 | 0 |
| | | (15.8) | | (7.8) | | (23.0) | | (7.4) | | (0.04) | | | | | |

This table describes the patient characteristics of our study patient populations [mean value +/− (standard deviation)]. dol: 10.1371/journal.pone.0012691.t001

TABLE 2

| | Primary | | | |
|---|---|---|---|---|
| Tissue Specimen | Diagnosis | Sex | Age | Race |
| Bladder | Bladder Cancer | M | 65 | Caucasian |
| Liver | Liver Cancer | M | 57 | Caucasian |
| Kidney | RCC | M | 71 | Caucasian |
| Colon | UC | M | 53 | Caucasian |
| Lung | Lung Cancer | M | 80 | Caucasian |
| Muscle | Sarcoma | M | 67 | Black |
| Pancreas | Cyst | F | 42 | Caucasian |
| Fat | Sarcoma | F | 81 | Black |
| Fat | Crohn's Disease | M | 50 | Caucasian |
| Brain | Epilepsy | M | 35 | Hispanic |
| Tonsil | Hypotrophy | F | 33 | Caucasian |
| Stomach | Unknown | M | 54 | Caucasian |
| Skin | Thigh Mass | M | 43 | Black |

Table 2 details the patient characteristics of the surgical specimens used for Western blotting and ELISA analysis, RCC = Renal Cell Carcinoma, UC = Ulcerative Colitis.
doi:10.1371/journal.pone.0012691.t002

TABLE 3

| Tissue Type | Proteins Detected | Nomenclature |
|---|---|---|
| Human Brain | S100B, S100A | S100 calcium binding protein A1 |
| | | (Acc# 5454032, 11 kDa) |
| | | S100 protein, beta polypeptide |
| | | (Acc# 5454034, 11 kDa) |
| Human Fat (F) | S100B, S100A | S100 calcium binding protein A1 |
| | | (Acc# 5454032, 11 kDa) |
| | | S100 protein, beta polypeptide |
| | | (Acc# 5454034, 11 kDa) |
| Human Fat (M) | S100B, S100A | S100 calcium binding protein A1 |
| | | (Acc# 5454032, 11 kDa) |
| | | S100 protein, beta polypeptide |
| | | (Acc# 5454034, 11 kDa) |

Mass spectrometry analysis revealed that human brain and fat tissue contained S100B as well as the presence of another protein of the S100 family, namely S100A.
doi:10,1371/journal.pone.0012591.t003

TABLE 4
Table 4. Summary of Patients' data

| | | Seizure | Type of | | | CGs/ | Dose | | Age at | Efficacy |
|---|---|---|---|---|---|---|---|---|---|---|
| ID | Sex | Type | Epilepsy | Etiology | AED | ACTH | kg/day | | treat. (y, m) | % |
| C.A.1 | F | Partial | EPC | mitocondrial | PB, VPA, TPM, | 2 | 1 | mg | 3 | 50 |
| | | | | disease | PHT, MDZ, | | | | | |
| C.A.2 | F | Partial | EPC | mitocondrial | PB, VPA, TPM, | 3 | 20 | mg | 3 | 30 |
| | | | | disease | LVT, PHT, MDZ, | | | | | |
| C.A.3 | F | Partial | EPC | mitocondrial | PB, VPA, TPM, | 3 | 20 | mg | 3 | 50 |
| | | | | disease | LVT, PHT, MDZ, | | | | | |
| | | | | | CZP | | | | | |
| C.L. | F | Partial | EPC | genetic non | CBZ, TPM, PB, | 3 | 15 | mg | 7 | 50 |
| | | | | progressive | VPA, PHT, GVG, | | | | | |
| | | | | | LTG, Clo, CZP, | | | | | |
| | | | | | LVT, MDZ, | | | | | |
| C.M. | F | Partial + SG | EPC | unknown | CBZ, PB, VPA, | 3 | 15 | mg | 5.9 | 50 |
| | | | | | Clo, CZP, LVT, | | | | | |
| | | | | | MDZ, ACZ | | | | | |
| C.M.1 | F | Partial | EPC | focal | PB, PHT, LTG, | 4 | 3 | mg | 4.9 | 100 |
| | | | | dysplasia | Clo, CBZ, VPA, | | | | | |
| | | | | | ESM, GVG, ACZ | | | | | |
| C.M.2 | F | Partial | EPC | focal | PB, PHT, LTG, | 2 | 0.44 | mg | 4.9 | 75 |
| | | | | dysplasia | Clo, CBZ, VPA, | | | | | |
| | | | | | ESM, GVG, ACZ | | | | | |
| D.C.1 | F | Partial + SG | EPC | unknown | CBZ, TPM, PB, | 3 | 15 | mg | 6.1 | 75 |
| | | | | | Clo, BR, LVT, | | | | | |
| | | | | | ZNS | | | | | |
| D.C.2 | F | Partial + SG | EPC | unknown | CBZ, TPM, PB, | 3 | 15 | mg | 7 | 0 |
| | | | | | Clo, BR, LVT, | | | | | |
| | | | | | ZNS | | | | | |
| D.C.3 | F | Partial + SG | EPC | unknown | CBZ, TPM, PB, | 3 | 15 | mg | 8.8 | 50 |
| | | | | | Clo, BR, LVT, | | | | | |
| | | | | | ZNS | | | | | |
| D.C.A | M | tonic-clonic/ | EPC | unknown | TPM, Pir, CZP, | 3 | 15 | mg | 14.1 | 30 |
| | | myoclonic/focal | | | CBZ, PB, Clo, | | | | | |
| M.T.V.1 | F | Partial | EPC | congenital | PB, TPM, GVG, | 1 | 3 | U | 0.6 | 0 |
| | | | | muscular | Clo, MDZ, PHT, | | | | | |
| | | | | dystrophy | BR | | | | | |
| M.T.V.2 | F | Partial | EPC | congenital | PB, TPM, GVG, | 2 | 0.2 | mg | 0.7 | 0 |
| | | | | muscular | Clo, MDZ, PHT, | | | | | |
| | | | | dystrophy | BR | | | | | |
| P.E.1 | M | Partial/SG | EPC | unknown/ | PB, VPA, PHT, | 2 | 0.45 | mg | 7.1 | 0 |
| | | | | progressive | CBZ, GVG, LTG, | | | | | |
| | | | | | Clo, CZP, LVT, | | | | | |
| | | | | | MDZ, Lor | | | | | |
| P.E.2 | M | Partial/SG | EPC | unknown/ | PB, VPA, PHT, | 2 | 0.2 | mg | 7.8 | 0 |
| | | | | progressive | CBZ, GVG, LTG, | | | | | |
| | | | | | Clo, CZP, LVT, | | | | | |
| | | | | | MDZ, Lor, TGB | | | | | |
| P.F. | F | Partial/SG | EPC | unknown | TPM, VPA, PHT, | 3 | 15 | mg | 8 | 50 |
| | | | | | Clo, LVT, CBZ | | | | | |
| V.J.1 | F | Myoclonic, | EPC | unknown/ | PB, VPA, PHT, | 3 | 15 | mg | 16 | 25 |
| | | partial | | progressive | CBZ, GVG, LTG, | | | | | |
| | | | | | Clo, CZP, LVT, | | | | | |
| | | | | | MDZ, PRM, TPM, | | | | | |
| V.J.2 | F | Myoclonic, | EPC | unknown/ | PB, VPA, PHT, | 3 | 15 | mg | 16.2 | 0 |
| | | partial | | progressive | CBZ, GVG, LTG, | | | | | |
| | | | | | Clo, CZP, LVT, | | | | | |
| | | | | | MDZ, PRM, TPM, | | | | | |
| B.C.1 | F | Partial/SG | Focal | chromo- | VPA, CZP, Clo, | 2 | 0.5 | mg | 0.8 | 100 |
| | | | | somopathy | ESM, LTG, PB, | | | | | |
| | | | | | GVG, CBZ, TPM | | | | | |
| B.C.2 | F | Partial/SG | Focal | chromo- | VPA, CZP, Clo, | 4 | 7.5 | mg | 0.9 | 100 |
| | | | | somopathy | ESM, LTG, PB, | | | | | |
| | | | | | GVG, CBZ, TPM | | | | | |
| B.L.1 | M | Partial/ | Focal | cerebral | PB, VPA, Clo, | 4 | 5 | mg | 6.3 | 75 |
| | | myoclonic | | palsy | CBZ, ESM, LVT, | | | | | |
| | | | | | LTG | | | | | |
| B.L.2 | M | Partial/ | Focal | cerebral | PB, VPA, Clo, | 4 | 5 | mg | 7.8 | 100 |
| | | myoclonic | | palsy | CBZ, ESM, LVT, | | | | | |
| | | | | | LTG | | | | | |
| B.L.3 | M | Partial/ | Focal | cerebral | PB, VPA, Clo, | 1 | 2.6 | U | 8 | 50 |
| | | myoclonic | | palsy | CBZ, ESM, LVT, | | | | | |
| | | | | | LTG | | | | | |
| B.V.1 | F | Partial/SG/ | Focal | unknown | PB, VPA, CBZ, | 1 | 3.6 | U | 1.3 | 100 |
| | | spasms | | | GVG, LTG, Clo, | | | | | |
| | | | | | CZP, LVT, PRM, | | | | | |
| | | | | | TPM, ESM, BR, | | | | | |
| | | | | | KD | | | | | |
| B.V.2 | F | Partial/SG/ | Focal | unknown | PB, VPA, CBZ, | 3 | 2 | mg | 3 | 0 |
| | | spasms | | | GVG, LTG, Clo, | | | | | |
| | | | | | CZP, LVT, PRM, | | | | | |
| | | | | | TPM, ESM, BR, | | | | | |
| | | | | | KD | | | | | |
| C.A. | M | Partial/SG | Focal | unknown | VPA, PB, CBZ, | 1 | 4.7 | U | 3.8 | 100 |
| | | | | | TPM, LTG | | | | | |
| C.F. | F | Partial/ | Focal | genetic non | B6, GVG, VPA, | 1 | 3 | U | 1.5 | 75 |
| | | myoclonic | | progressive | LTG, Clo | | | | | |
| C.L.1 | M | Partial/ | Focal | genetic non | CZP, VPA, Clo, | 4 | 10 | mg | 3 | 100 |
| | | myoclonic | | progressive | GVG, ESM, CBZ, | | | | | |
| | | | | | LTG, MSM | | | | | |
| C.L.2 | M | Partial/ | Focal | genetic non | CZP, VPA, Clo, | 4 | 10 | mg | 3.5 | 100 |
| | | myoclonic | | progressive | GVG, ESM, CBZ, | | | | | |
| | | | | | LTG, MSM | | | | | |
| C.L.3 | M | Partial/ | Focal | genetic non | CZP, VPA, Clo, | 4 | 5 | mg | 7.2 | 100 |
| | | myoclonic | | progressive | GVG, ESM, CBZ, | | | | | |
| | | | | | LTG, MSM | | | | | |
| C.M.1 | F | Partial/SG | Focal | focal | VPA, ESM, LVT, | 2 | 0.1 | mg | 6.9 | 90 |
| | | | | dysplasia | Clo, TPM, BR, | | | | | |
| | | | | | CBZ, PHT, PB | | | | | |
| C.M.2 | F | Partial | Focal | focal | VPA, ESM, LVT, | 2 | 0.1 | mg | 6.9 | 70 |
| | | | | dysplasia | Clo, TPM, BR, | | | | | |
| | | | | | CBZ, PHT, PB | | | | | |
| C.M.3 | F | Partial | Focal | focal | VPA, ESM, LVT, | 4 | 8 | mg | 8.4 | 50 |
| | | | | dysplasia | Clo, TPM, BR, | | | | | |
| | | | | | CBZ, PHT, PB | | | | | |
| C.M.4 | F | Partial/SG | Focal | focal | VPA, ESM, LVT, | 4 | 8.3 | mg | 8.4 | 0 |
| | | | | dysplasia | Clo, TPM, BR, | | | | | |
| | | | | | CBZ, PHT, PB | | | | | |
| C.R.C.1 | F | Partial + SG | Focal | focal | VPA, GVG, CBZ, | 2 | 0.4 | mg | 5.8 | 50 |
| | | | | dysplasia | LTG, Clo, TPM, | | | | | |
| C.R.C.2 | F | Partial + SG | Focal | focal | VPA, GVG, CBZ, | 2 | 0.4 | mg | 6 | 50 |
| | | | | dysplasia | LTG, Clo, TPM, | | | | | |
| C.R.C.3 | F | Partial + SG | Focal | focal | VPA, GVG, CBZ, | 2 | 1 | mg | 8.6 | 70 |
| | | | | dysplasia | LTG, Clo, TPM, | | | | | |
| | | | | | LVT, ZNS, RFN, | | | | | |
| | | | | | PB | | | | | |
| D.M.A | F | Partial with | Focal | unknown | CBZ, LVT, Clo, | 3 | 15 | mg | 13 | 70 |
| | | sporadic | | | PB, MDZ, Lor, | | | | | |
| | | status | | | SUL, PHT, | | | | | |
| F.A. | M | Partial/ | Focal | Krabbe | VPA, ESM, LTG | 2 | 0.2 | mg | 7.2 | 80 |
| | | tonic | | disease, | | | | | | |
| | | | | stem cell | | | | | | |
| | | | | transplantation | | | | | | |
| I.N. | F | Myoclonic, | Focal | Krabbe | VPA, CBZ, PRM, | 3 | 10 | mg | 8.8 | 100 |
| | | partial | | disease, | LVT, PB | | | | | |
| | | | | stem cell | | | | | | |
| | | | | transplantation | | | | | | |
| M.T. | M | Partial | Focal | focal | | 3 | 30 | mg | 3.9 | 0 |
| | | | | dysplasia | | | | | | |
| R.S.1 | F | Partial | Focal | focal | GVG, PHT, PB, | 2 | 0.3 | mg | 0.5 | 80 |
| | | | | dysplasia | PRM, Clo, CBZ, | | | | | |
| | | | | | VPA | | | | | |
| R.S.2 | F | Partial | Focal | focal | GVG, PHT, PB, | 4 | 10 | mg | 1 | 80 |
| | | | | dysplasia | PRM, Clo, CBZ, | | | | | |
| | | | | | VPA | | | | | |
| V.A. | M | Partial/SG | Focal | diffuse | PB, VPA, PHT, | 1 | 2.6 | U | 14 | 100 |
| | | | | cortical | GVG, LTG, BR, | | | | | |
| | | | | dysplasia | Clo, LVT, TPM, | | | | | |
| | | | | | KD | | | | | |
| V.E. | F | Partial + SG | Focal | unknown | CBZ, VPA, Clo, | 2 | 0.2 | g | 17 | 80 |
| | | | | | LTG, GVG, PHT, | | | | | |
| | | | | | PRM, PB, FBM, | | | | | |
| | | | | | LVT, TPM, | | | | | |
| V.M.1 | M | Partial/ | Focal | unknown | PB, TPM, CBZ, | 4 | 10 | mg | 1.3 | 30 |
| | | tonic/ | | | LTG, GVG, PRM, | | | | | |
| | | absences | | | FBM, VPA, ESM, | | | | | |
| | | | | | PHT, CBZ, Clo, | | | | | |
| | | | | | ZNS | | | | | |
| V.M.2 | M | Partial/ | Focal | unknown | PB, TPM, CBZ, | 1 | 4 | U | 2.7 | 30 |
| | | tonic/ | | | LTG, GVG, PRM, | | | | | |
| | | absences | | | FBM, VPA, ESM, | | | | | |
| | | | | | PHT, CBZ, Clo, | | | | | |
| | | | | | ZNS | | | | | |
| V.M.3 | M | Partial/ | Focal | unknown | PB, TPM, CBZ, | 1 | 2 | U | 13.8 | 30 |
| | | tonic/ | | | LTG, GVG, PRM, | | | | | |
| | | absences | | | FBM, VPA, ESM, | | | | | |
| | | | | | PHT, CBZ, Clo, | | | | | |
| | | | | | ZNS | | | | | |
| M.M. | M | Partial/ | Focal | cerebral | CBZ, GVG, VPA, | 2 | 0.1 | mg | 18.3 | 60 |
| | | myoclonic | | palsy | Clo, ESM, LTG, | | | | | |
| | | | | | PRM, PHT, TPM | | | | | |
| P.E. | F | Partial | Focal | unknown/ | CBZ, TPM, PB, | 3 | 15 | mg | 14 | 100 |
| | | multifocal | | non | LTG, Clo, GVG, | | | | | |
| | | | | progressive | MDZ | | | | | |
| C.G.1 | M | Myoclonic, | Generalized | mitocondrial | CBZ, VPA, Clo, | 1 | 0.7 | U | 14.7 | 70 |
| | | absence, SG | | disease | FBM, ESM, LVT, | | | | | |
| | | | | | PRM, NTZ | | | | | |
| C.G.2 | M | Myoclonic, | Generalized | mitocondrial | CBZ, VPA, Clo, | 1 | 1.5 | U | 14.8 | 70 |
| | | absence, SG | | disease | FBM, ESM, LVT, | | | | | |
| | | | | | PRM, NTZ | | | | | |
| C.G.3 | M | Myoclonic, | Generalized | mitocondrial | CBZ, VPA, Clo, | 4 | 4.2 | mg | 15.3 | 70 |
| | | absence, SG | | disease | FBM, ESM, LVT, | | | | | |
| | | | | | PRM, NTZ | | | | | |
| C.G.4 | M | Myoclonic, | Generalized | mitocondrial | CBZ, VPA, Clo, | 1 | 1.5 | U | 15.6 | 70 |
| | | absence, SG | | disease | FBM, ESM, LVT, | | | | | |
| | | | | | PRM, NTZ | | | | | |
| C.G.5 | M | Myoclonic, | Generalized | mitocondrial | CBZ, VPA, Clo, | 1 | 1.35 | U | 18.4 | 70 |
| | | absence, SG | | disease | FBM, ESM, LVT, | | | | | |
| | | | | | PRM, NTZ | | | | | |
| C.G.6 | M | Myoclonic, | Generalized | mitocondrial | CBZ, VPA, Clo, | 1 | 1.35 | U | 22 | 70 |
| | | absence, SG | | disease | FBM, ESM, LVT, | | | | | |
| | | | | | PRM, NTZ | | | | | |
| I.P. | M | Generalized | Generalized | unknown | VPA, Clo, ESM, | 1 | 2.9 | U | 3.1 | 30 |
| | | myoclonic | | | LTG, LVT | | | | | |
| V.A. | F | myoclonic | Generalized | genetic non | VPA, ESM | 1 | 3 | U | 2.7 | 50 |
| | | | | progressive | | | | | | |
| S.A.1 | F | Partial | MMPEI | unknown | PB, GVG, PHT, | 2 | 0.65 | mg | 0.6 | 35 |
| | | (multifocal) | | | VPA, CBZ, CNZ | | | | | |
| S.A.2 | F | Partial | MMPEI | unknown | PB, GVG, PHT, | 2 | 0.65 | mg | 0.6 | 35 |
| | | (multifocal) | | | VPA, CBZ, CNZ | | | | | |
| S.A.3 | F | Partial | MMPEI | unknown | PB, GVG, PHT, | 2 | 0.75 | mg | 0.6 | 35 |
| | | (multifocal) | | | VPA, CBZ, CNZ | | | | | |
| S.A.4 | F | Partial | MMPEI | unknown | PB, GVG, PHT, | 2 | 0.75 | mg | 0.6 | 35 |
| | | (multifocal) | | | VPA, CBZ, CNZ | | | | | |
| S.A.5 | F | Partial | MMPEI | unknown | PB, GVG, PHT, | 2 | 0.75 | mg | 0.6 | 35 |
| | | (multifocal) | | | VPA, CBZ, CNZ | | | | | |
| S.A.6 | F | Partial | MMPEI | unknown | PB, GVG, PHT, | 2 | 0.75 | mg | 0.9 | 35 |
| | | (multifocal) | | | VPA, CBZ, CNZ | | | | | |
| S.A.7 | F | Partial | MMPEI | unknown | PB, GVG, PHT, | 2 | 0.75 | mg | 0.9 | 35 |
| | | (multifocal) | | | VPA, CBZ, CNZ | | | | | |
| S.A.8 | F | Partial | MMPEI | unknown | PB, GVG, PHT, | 2 | 0.75 | mg | 0.9 | 100 |
| | | (multifocal) | | | VPA, CBZ, CNZ | | | | | |
| S.A.9 | F | Partial | MMPEI | unknown | PB, GVG, PHT, | 2 | 0.75 | mg | 0.9 | 35 |
| | | (multifocal) | | | VPA, CBZ, CNZ | | | | | |
| S.A.10 | F | Partial | MMPEI | unknown | PB, GVG, PHT, | 2 | 0.75 | mg | 0.9 | 35 |
| | | (multifocal) | | | VPA, CBZ, CNZ | | | | | |
| Z.R.1 | M | Partial | MMPEI | unknown | PB, ACTH, GVG, | 4 | 10 | mg | 2.1 | 70 |
| | | (multifocal) | | | LTG, CBZ, VPA, | | | | | |
| | | | | | CNZ, BR | | | | | |
| Z.R.2 | M | Partial | MMPEI | unknown | PB, ACTH, GVG, | 2 | 0.2 | mg | 2.8 | 50 |
| | | (multifocal) | | | LTG, CBZ, VPA, | | | | | |
| | | | | | CNZ, BR | | | | | |
| A.F. | M | Myoclonic, | non conv. | genetic non | VPA, Clo, LVT, | 1 | 3.5 | U | 4.1 | 100 |
| | | partial | ES | progressive | LTG | | | | | |
| A.S. | F | Partial | non conv. | diffuse | VPA, ESM, Clo, | 1 | 3 | U | 6 | 90 |
| | | (multifocal) | ES | cortical | LTG | | | | | |
| | | | | dysplasia | | | | | | |
| C.C.1 | F | Tonic/ | non conv. | unknown | PB, VPA, Clo, | 1 | 5 | U | 0.4 | 80 |
| | | spasms/focal | ES | | GVG, TPM, LTG, | | | | | |
| | | | | | DZP, CNZ, BR, | | | | | |
| | | | | | LVT | | | | | |
| C.C.2 | F | Tonic/ | non conv. | unknown | PB, VPA, Clo, | 4 | 5 | mg | 4.5 | 25 |
| | | spasms/focal | ES | | GVG, TPM, LTG, | | | | | |
| | | | | | DZP, CNZ, BR, | | | | | |
| | | | | | LVT | | | | | |
| C.C.3 | F | Tonic/ | non conv. | unknown | PB, VPA, Clo, | 1 | 2.6 | U | 6.9 | 75 |
| | | spasms/focal | ES | | GVG, TPM, LTG, | | | | | |
| | | | | | DZP, CNZ, BR, | | | | | |
| | | | | | LVT | | | | | |
| C.F.1 | M | Partial | non conv. | unknown | CBZ, VPA, TPM, | 3 | 15 | mg | 4.6 | 20 |
| | | | ES | | LVT, Clo, MDZ, | | | | | |
| | | | | | ESM, DZP, LTG | | | | | |
| C.F.2 | M | Partial | non conv. | unknown | CBZ, VPA, TPM, | 1 | 2.5 | U | 4.6 | 0 |
| | | | ES | | LVT, Clo, MDZ, | | | | | |
| | | | | | ESM, DZP | | | | | |
| G.M.1 | F | Partial/ | non conv. | Post- | PB, CZP, CBZ, | 3 | 10 | mg | 16 | 50 |
| | | myoclonic | ES | surgical | PRM, VPA, ESM, | | | | | |
| | | | | | LTG | | | | | |
| G.M.2 | F | Partial/ | non conv. | Post- | PB, CZP, CBZ, | 3 | 15 | mg | 16 | 20 |
| | | myoclonic | ES | surgical | PRM, VPA, ESM, | | | | | |
| | | | | | LTG | | | | | |
| M.M. | M | tonic | non conv. | genetic non | VPA, ESM, LTG, | 1 | 2.5 | U | 6 | 100 |
| | | | ES | progressive | CZP, LVT, SUL, | | | | | |
| | | | | | TPM, | | | | | |
| M.P.1 | M | Partial/ | non conv. | genetic non | VPA, ESM, Clo, | 1 | 3 | U | 4 | 80 |
| | | myoclonic | ES | progressive | LVT, TPM, LTG, | | | | | |
| | | | | | CNZ, BR, PB | | | | | |
| M.P.2 | M | Myoclonic | non conv. | genetic non | VPA, ESM, Clo, | 1 | 3 | U | 4.3 | 75 |
| | | | ES | progressive | LVT, TPM, LTG, | | | | | |
| | | | | | CNZ, BR, PB | | | | | |
| M.P.3 | M | Myoclonic | non conv. | genetic non | VPA, ESM, Clo, | 1 | 3 | U | 4.8 | 0 |
| | | | ES | progressive | LVT, TPM, LTG, | | | | | |
| | | | | | CNZ, BR, PB | | | | | |
| M.P.4 | M | Partial/ | non conv. | genetic non | VPA, ESM, Clo, | 1 | 3 | U | 5 | 0 |
| | | myoclonic | ES | progressive | LVT, TPM, LTG, | | | | | |
| | | | | | CNZ, BR, PB | | | | | |
| N.M.1 | M | Partial | non conv. | diffuse | VPA, CBZ, LTG, | 4 | 10 | mg | 2 | 75 |
| | | | ES | cortical | PHT, TPM, PB, | | | | | |
| | | | | dysplasia | ESM | | | | | |
| N.M.2 | M | Partial | non conv. | diffuse | VPA, CBZ, LTG, | 4 | 10 | mg | 2.4 | 50 |
| | | | ES | cortical | PHT, TPM, PB, | | | | | |
| | | | | dysplasia | ESM | | | | | |
| S.K.1 | M | myoclonic | non conv. | genetic non | CPA, CNZ, PB, | 1 | 2.6 | U | 5.9 | 100 |
| | | | ES | progressive | LVT, ESM, Clo, | | | | | |
| | | | | | LTG, VPA | | | | | |
| S.K.2 | M | myoclonic | non conv. | genetic non | CPA, CNZ, PB, | 4 | 5 | mg | 6.5 | 10 |
| | | | ES | progressive | LVT, ESM, Clo, | | | | | |
| | | | | | LTG, VPA | | | | | |
| S.K.3 | M | myoclonic | non conv. | genetic non | CPA, CNZ, PB, | 4 | 2.5 | mg | 6.9 | 75 |
| | | | ES | progressive | LVT, ESM, Clo, | | | | | |
| | | | | | LTG, VPA | | | | | |

| | | | | | | | | | |
|---|---|---|---|---|---|---|---|---|---|
| V.C. | F | Partial/SG | non conv. | genetic non | VPA, Clo, TPM, | 2 | 0.15 | 7 | 80 |
| | | | ES | progressive | LVT, CBZ, GVG | | | | |

1 = ACTH; 2 = dexamethasone; 3 = metilprednisolone; 4 = hydrocortisone
MMPE: malignant migrating partial epilepsy of infancy; SG = secondarily generalized
VPA = valproic acid; PRM = primidone; ESM = Etosuximide; LTG = Lamotrigine; PB = Phenobarbital; CBZ = Carbamazepine; PHT = Phenitoin; MDZ = Midazolam; Clo = Clobazam; CNZ = Clonazepam; NTZ = nitrazepam; SUL = Sulthiame; GVG = vigabatrin; FBM = felbamate; ZNS = zonisamide. AEDs co-administered with GCs or ACTH are indicated in bold.
KD = Ketogenic diet; BR = Bromides

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of assessing blood brain barrier permeability in an individual comprising
a) selectively detecting a level of S100BB homodimer in a sample of the individual, and
b) comparing the level of S100BB homodimer in the sample to a level of S100B a control,
wherein an elevated level of S100BB homodimer in the sample compared to the level of S100BB homodimer in the control, is indicative of blood brain barrier permeability in the individual.

2. The method of claim 1 wherein the S100BB homodimer is detected using an immunoassay.

3. The method of claim 2 wherein the immunoassay is an immunoprecipitation assay.

4. The method of claim 1 further comprising detecting one or more levels of one or more markers of neuronal distress.

5. The method of claim 4 wherein the one or more markers of neuronal distress comprise Ubiquitin C-terminal hydrolase 1, NSE, GFAP, tau protein, beta trace protein, cystatin C.

6. The method of claim 1 further comprising detecting auto-antibodies directed against S100B, S100BB, S100AB or a combination thereof in a sample of the individual.

7. A method for delivering an agent for delivery to the brain of an individual in need thereof comprising:
a) introducing a first agent that opens the blood brain barrier into the individual;
b) selectively determining the level of S100BB homodimer in a sample of the individual, wherein an elevated level of S100BB homodimer in the sample compared to the level of S100BB homodimer in the control, indicates that the blood brain barrier of the individual is permeable to the agent for delivery to the brain; and
c) introducing the agent for delivery to the brain to the individual when the blood brain barrier of the individual is permeable, thereby delivering the agent for delivery to the brain of the individual.

8. The method of claim 7 wherein the S100BB homodimer is detected using an immunoassay.

9. The method of claim 8 wherein the immunoassay is an immunoprecipitation assay.

10. The method according to claim 7 wherein the agent for delivery to the brain is introduced into the individual's bloodstream in a vicinity of the individual's brain.

11. The method according to claim 7, wherein the agent to be delivered to the brain is a contrast agent, a neuropharmacologic agent, a neuroactive peptides, a protein, an enzyme, a gene therapy agent, a neuroprotective factor, a growth factor, a biogenic amine, a trophic factor to any of brain and spinal transplants, an immunoreactive proteins, a receptor binding protein, a radioactive agent, an antibody, a cytotoxin or a combination thereof.

12. A method of detecting whether a cancer has metastasized to a cancer patient's brain in a patient that has, or is at risk of having, metastasis, comprising
a) detecting a level of S100B in a sample of the cancer patient using an immunoassay, and
b) detecting a level of S100B in a sample using an immunoassay that differs from the immunoassay in a), and
c) comparing the level of S100B of a) and b) to a level of S100B in a control, wherein if the level of S100B in a) and the level of S100B in b) are the same as, or lower than the level of S100B in the control then, the metastasis has not spread to the cancer patient's brain.

13. The method of claim 12 wherein the level of S100B is the level of S100B monomer, S100BB homodimer, S100AB or total S100B.

14. The method of claim 12 wherein the level of S100BB homodimer is selectively detected.

15. The method of claim 12 wherein at least one immunoassay is an immunoprecipitation assay.

16. A method of determining the effectiveness of a treatment for a neurological disorder wherein blood-brain barrier permeability is present in an individual in need thereof comprising
a) detecting a level of S100B in a sample of the individual undergoing the treatment, and
b) comparing the level of S100B in the sample to the level of S100B in a sample from the individual obtained prior to treatment, wherein decreased levels of S100B in the sample compared to the level of S100B in the sample from the individual obtained prior to treatment indicate that the treatment for a neurological disorder wherein blood-brain barrier permeability is present is effective in the individual.

17. The method of claim 16 wherein the level of S100B is the level of S100B monomer, S100BB homodimer, S100AB heterodimer, total S100B or a combination thereof.

18. The method of claim 16 further comprising obtaining a sample from the individual prior to treatment and detecting the level of S100B in the sample.

19. The method of claim 16 wherein the S100B is detected using an immunoassay.

20. The method of claim 19 wherein the immunoassay is an immunoprecipitation assay.

21. A method of determining the effectiveness of a treatment for seizures triggered by blood brain barrier damage in an individual in need thereof comprising
a) detecting a level of S100B in a sample of the individual undergoing the treatment, and
b) comparing the level of S100B in the sample to the level of S100B in a sample from the individual obtained prior to treatment, wherein decreased levels of S100B in the sample compared to the level of S100B in the sample from the individual obtained prior to treatment indicate that the treatment is effective to treat the seizures in the individual.

22. The method of claim 21 wherein the level of S100B is the level of S100B monomer, S100BB homodimer, S100AB heterodimer, total S100B or a combination thereof.

23. The method of claim 21 further comprising obtaining a sample from the individual prior to treatment and detecting the level of S100B in the sample.

24. The method of claim 21 wherein the S100B is detected using an immunoassay.

25. The method of claim 24 wherein the immunoassay is an immunoprecipitation assay.

26. A method of determining the effectiveness of a hypothermia treatment in an individual in need thereof comprising
a) detecting a level of S100B in a sample of the individual undergoing the hypothermia treatment, and
b) comparing the level of S100B in the sample to the level of S100B in a sample from the individual obtained prior to hypothermia treatment, wherein decreased levels of S100B in the sample compared to the level of S100B in the sample from the individual obtained prior to treatment indicate that the hypothermia treatment is effective to treat the individual.

27. The method of claim 26 wherein the level of S100B is the level of S100B monomer, S100BB homodimer, S100AB heterodimer, total S100B or a combination thereof.

28. The method of claim 26 further comprising obtaining a sample from the individual prior to the hypothermia treatment and detecting the level of S100B in the sample.

29. The method of claim 26 wherein the hypothermia is administered to treat ischemic-hemorrhagic stroke, to mitigate seizures or during a surgical cardiac procedure in the individual.

30. The method of claim 26 wherein the S100B is detected using an immunoassay.

31. The method of claim 30 wherein the immunoassay is an immunoprecipitation assay.

32. A method of detecting a positive outcome for a newborn that has undergone asphyxia during birth comprising
a) detecting a level of S100B in a sample of the newborn at birth,
b) detecting at least one level of S100B in one or more samples of the newborn after birth, and
c) comparing the at least one level of S100B in the sample after birth to the level of S100B in the sample at birth, wherein a decreased level of S100B in the sample after birth compared to the level of S100B at birth indicate a positive outcome for the newborn.

33. The method of claim 32 wherein the level of S100B is the level of S100B monomer, S100BB homodimer, S100AB heterodimer, total S100B or a combination thereof.

34. The method of claim 32 further comprising obtaining one or more samples from the newborn.

35. The method of claim 32 wherein the S100B is detected using an immunoassay.

36. The method of claim 35 wherein the immunoassay is an immunoprecipitation assay.

37. A method of detecting a sub-concussion in an individual in need thereof comprising
a) detecting a level of S100B in a sample of the individual, and
b) comparing the level of S100B in the sample to a level of S100B a control, wherein elevated levels of S100B in the sample compared to the level of S100B in the control indicate that the individual has a sub-concussion.

38. The method of claim 37 wherein the individual has had one or more concussions, sub-concussions, seizures or a combination thereof.

39. The method of claim 37 wherein the level of S100B is the level of S100B monomer, S100BB homodimer, S100AB heterodimer, S100B total or a combination thereof.

40. The method of claim 37 wherein the S100B is detected using an immunoassay.

41. The method of claim 40 wherein the immunoassay is an immunoprecipitation assay.

42. A method of detecting a history of blood brain barrier disruption in an individual in need thereof comprising detecting auto-antibodies directed against S100B in a sample of the individual, wherein the presence of auto-antibodies directed against S100B in the sample indicates that the individual has a history of blood brain barrier disruption.

43. The method of claim 42 wherein the auto-antibodies are directed against S100B monomer, S100BB heterodimer, S100AB heterodimer or a combination thereof.

44. The method of claim 42 further comprising detecting a level of S100B in a sample of the individual wherein elevated levels of S100B in the sample compared to the level of S100B in the control further indicates that the individual has a history of blood brain barrier disruption.

45. The method of claim 44 wherein the individual has ongoing blood brain barrier disruption.

46. The method of claim 42 wherein the individual has an increased risk for degenerative brain disease.

47. The method of claim 42 wherein the individual has had one or more concussion, sub-concussions, seizures or a combination thereof.

48. The method of claim 42 further comprising obtaining a sample from the individual.

49. The method of claim 42 wherein the auto-antibodies are detected using en enzyme-linked immunosorbent assay (ELISA).

50. The method of claim 44 wherein the level of S100B is the level of S100B monomer, S100BB homodimer, S100AB heterodimer, S100B total or a combination thereof.

51. The method of claim 42 wherein the S100B is detected using an immunoassay.

52. The method of claim 51 wherein the immunoassay is an immunoprecipitation assay.
